(19) United States
(12) Patent Application Publication
(10) Pub. No.: US 2008/0175800 A1
(43) Pub. Date: Jul. 24, 2008

(54) USE OF SPECIFIC MENTHYL 3-OXOCARBOXYLIC ACID ESTERS AS PHYSIOLOGICALLY ACTIVE COOLING SUBSTANCES

(75) Inventors: Axel Schoening, Holzminden (DE); Bernd Wiedwald, Holzminden (DE)

Correspondence Address: CONNOLLY BOVE LODGE & HUTZ, LLP, P O BOX 2207, WILMINGTON, DE, 19899, US

(73) Assignee: SYMRISE GmbH & Co. KG, Holzminden (DE)

(21) Appl. No.: 11/969,554

(22) Filed: Jan. 4, 2008

Related U.S. Application Data
(60) Provisional application No. 60/883,400, filed on Jan. 4, 2007.

(51) Int. Cl. A61K 8/37; A61K 31/215; A01N 37/02; A01P 7/04; A61Q 13/00; A61Q 15/00; A61Q 11/00; A61Q 9/00; A61Q 19/10

(52) U.S. Cl. 424/49; 514/546; 424/59; 424/73; 424/70.1; 424/70.2; 424/70.6; 424/61; 424/65; 424/63; 424/64; 510/130; 510/159; 510/119; 512/23

(57) ABSTRACT

The use is described of a compound of the formula (I) or (ent-I) or a blend consisting of two, three or more compounds of the formula (I) or (ent-I)

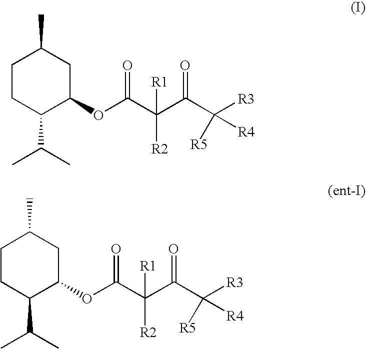

(a) as a cooling substance for non-therapeutic purposes or
(b) for the production of a medicament,
wherein in each of the formulae (I) and (ent-I)
R1, R2, R3, R4 and R5 mutually independently in each case mean hydrogen or a linear, branched or cyclic, saturated or unsaturated hydrocarbon residue having 1 to 4 carbon atoms.

### RELATED APPLICATIONS

This application claims benefit of U.S. Provisional Application 60/883,400, filed Jan. 4, 2007.

The present invention relates to the use of specific menthyl 3-oxocarboxylic acid esters of the formulae (I) and/or (ent-I) stated further below as (physiologically active) cooling substances for non-therapeutic and therapeutic purposes and for producing medicaments. The invention additionally relates to the use of corresponding blends and specific novel blends comprising compounds of the formulae (I) or (ent-I) stated further below. The invention additionally relates to specific preparations which comprise a quantity of a compound of the formula (I) or (ent-I) or of a corresponding blend which is sufficient to achieve a physiological cooling action on the skin and/or mucous membranes. Finally the invention also relates to therapeutic and non-therapeutic methods for achieving a physiological cooling action on the skin and/or mucous membranes.

Hereinafter, the term “cooling substance” is in particular used to designate physiologically active cooling substances (cooling active ingredients).

Cooling substances are often used to bring about a sensation of coolness on the skin or mucous membranes, for example on the mucous membranes in the oral, nasal and/or pharyngeal cavities, but without any physical cooling, such as occurs for example on solvent evaporation, actually occurring. Both individual components and mixtures may be used as cooling substances.

The best known cooling substance is L-menthol, but this exhibits various disadvantages, for example a strong odor impression, elevated volatility and, at relatively high concentrations, a bitter and/or spicy hot intrinsic flavor. In certain (aroma) compositions, in particular those which do not tend towards a (pepper)mint aroma, the use of L-menthol may thus be undesirable.

J. Soc. Cosmet. Chem. 1978, 29, 185-200 presented the results of a study of approx. 1200 compounds, in which the compounds L-menthane carboxylic acid N-ethylamide (“WS3”) and in particular N^{α}-(L-menthanecarbonyl)glycine ethyl ester (“WS5”) were found to be the most strongly cooling substances. The latter, while having a strong action, has the disadvantage of being susceptible to hydrolysis and, as a result, forming the corresponding free acid N^{α}-(L-menthanecarbonyl)glycine, which itself retains only a very weak cooling action. Despite the exhaustive investigations which have been described, a systematic prediction of the properties of potential cooling substances, in particular regarding the bitterness thereof and/or the other trigeminal effects thereof, is not possible and has also not been described. Accordingly, while many molecules failing within the class of menthane carboxamides are indeed strongly cooling, they frequently simultaneously exhibit marked bitter notes (for example the menthane carboxylic acid N-(alkyloxyalkyl)amides according to JP 2004059474) or are additionally strongly irritant (WS5: N-[[5-methyl-2-(1-methylethyl)cyclohexyl]carbonyl]glycine ethyl ester, US 2005/0222256).

N^{α}-(Menthanecarbonyl)alkyloxyalkylamides have been described in JP 2004059474. These have a strong cooling action and elevated resistance to hydrolysis, but suffer the disadvantage of being strongly bitter and thus being unusable in foodstuffs and also in cosmetic products for facial care.

FR 2 577 922 discloses the production of L-menthyl 3-hydroxybutyrate and the use thereof as a cooling substance. This compound is produced by reducing the keto group of L-menthyl 3-oxobutyrate (compound of the formula (I-X), below). L-menthyl 3-oxobutyrate is in turn obtained according to FR 2 577 922 by means of esterification from menthol and diketene; no mention of the cooling action of L-menthyl 3-oxobutyrate is made therein. The present invention does not include the reaction mixtures according to FR 2 577 922.

Agric. Biol. Chem. 1983, 47, 1689-1690 discloses the synthesis of (L)-menthyl 3-oxohexanoate ((compound of the formula (I) with R1 to R4=H, R5=ethyl, below) from ethyl bromide and 1-menthyl acetoacetate (compound of the formula (I-X), below). (L)-Menthyl 3-oxohexanoate is used as a synthesis intermediate; no information is provided regarding a cooling action of the disclosed compounds.

DE 38 16 360 and DE 38 16 361 describe (−)-menthyl compounds of the formula (I) stated below with R1 and R2=H. These compounds are used as starting materials for producing pharmacologically active 1,4-dihydropyridine dicarboxylic acid (−)-menthyl esters. No cooling action of the disclosed compounds is described.

The primary object of the present invention was therefore to provide compounds or mixtures of compounds which have a strong physiological cooling action, stability (resistance to hydrolysis) which is good and improved in comparison with known cooling active ingredients, and which may be used as cooling substances (cooling active ingredients) in foodstuffs and/or products consumed for pleasure and/or oral care products and/or oral pharmaceutical preparations and/or cosmetic preparations. The compounds or mixtures of compounds to be provided should preferably exhibit the weakest possible intrinsic flavor, in particular should taste only slightly or not at all bitter and exhibit the slightest possible irritancy.

The primary object is achieved according to the invention by using

of a compound of the formula (I) or (ent-I) or
of a blend consisting of two, three or more compounds of the formula (I) or (ent-I)

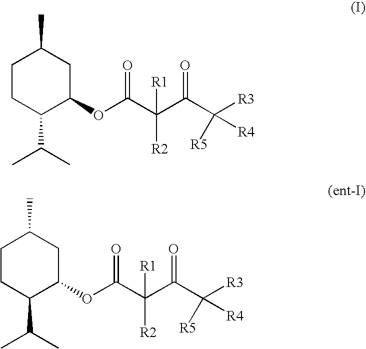

(a) as a cooling substance for non-therapeutic and therapeutic purposes or
(b) for the production of a medicament (in particular of a medicament having a physiological cooling action),
wherein in each of the formulae (I) and (ent-I), R1, R2, R3, R4 and R5 mutually independently in each case mean hydrogen or a linear, branched or cyclic, saturated or unsaturated hydrocarbon residue (residue solely comprising C and H atoms) with 1 to 4 carbon atoms.

The compounds of the formulae (I) and (ent-I) are hereinafter also designated menthyl 3-oxocarboxylic acid esters.

Within the group of compounds to be used according to the invention of the formulae (I) or (ent-I) or the corresponding blends, certain compounds are preferred. In particular, it is preferred for the compound or at least one of the compounds in the blend to be selected from the group consisting of compounds of the formulae (I) and (ent-I), wherein R1, R2, R3, R4 and R5 mutually independently in each case mean hydrogen or a methyl, ethyl, propyl, cyclopropyl, 2-propyl, 2-propenyl, 1-propenyl, 2-methylpropyl, methyl-2-propenyl, cyclobutyl, 1-butyl, 2-butyl, tert.-butyl or cyclopropylmethyl residue.

The compound or at least one of the compounds in the blend is here preferably selected from the group consisting of compounds of the formulae (I) and (ent-I), wherein R1 and R2 mean hydrogen. If R1 and R2 mean hydrogen, R3, R4, and R5 preferably mean a linear or branched, preferably saturated hydrocarbon residue with 1 to 4 carbon atoms, preferably methyl, ethyl, propyl, cyclopropyl, 2-propyl, 2-propenyl, 1-propenyl, 2-methylpropyl, methyl-2-propenyl, 1-butyl, 2-butyl, tert.-butyl, cyclopropylmethyl.

The compound (which is preferably stated above to be preferred) or at least one of the compounds (which are preferably stated above to be preferred) in the blend is/are preferably selected from the group consisting of compounds of the formulae (I) and (ent-I), wherein R3 and R4 mean hydrogen and R5 means hydrogen or methyl.

The compound or one of the compounds in the blend is particularly preferably selected from the group consisting of compounds of the formulae (I) and (ent-I), wherein R1, R2, R3 and R4 mean hydrogen and R5 means hydrogen or methyl.

Very particularly preferred individual compounds are thus compounds of the formulae (I) and (ent-I) with R1, R2, R3, R4=H and R5=H or CH₃, i.e.

L-menthyl 3-oxobutyrate (I-X)

### (R1, R2, R3, R4, R5=H)

L-menthyl 3-oxopentanoate (I-XX)

### (R1, R2, R3, R4=H; R5=CH₃)

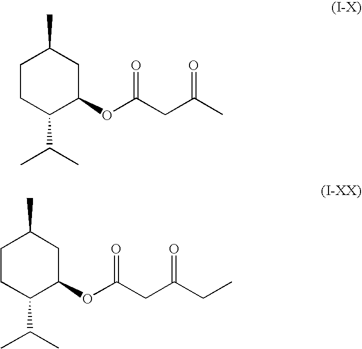

and the corresponding compounds with the configuration according to formula (ent-I).

The stated individual compounds thus alternatively assume the configuration according to formula (I) or (ent-I); they have not hitherto been described in the literature as cooling substances.

The blends to be used according to the invention consist of two, three or more compounds of the formulae (I) or (ent-I), preferably in each case in one of the developments which are stated above to be preferred. The presence of a compound of the formula (I) in addition to a compound of the formula (ent-I), wherein the meaning of the particular groups R1, R2, R3, R4 and R5 is identical in the formulae (I) and (ent-I) respectively, is particularly preferred in many cases; the presence of enantiomeric pairs is particularly preferred. It is moreover preferred if a blend to be used according to the invention contains not just one compound of the formula (I) or (ent-I) which is stated above to be particularly preferred, i.e. not just one compound which comprises one, several or solely particularly preferred groups R1, R2′ R3, R4 and R5, but instead two or more compounds of the formulae (I) and/or (ent-I) which are particularly preferred according to the invention. Two, three or all the compounds in a blend to be used according to the invention which consists of compounds of the formula (I) or (ent-I) are thus preferably selected from the group of compounds which are stated to be preferred.

The above explanations apply mutatis mutandis to the further aspects of the present invention stated below. A further aspect of the present invention relates to a blend consisting of or comprising:

(a) a first compound of the formula (I) or (ent-I) as defined above, in particular in one of developments which are stated above to be preferred,
and one or more substances selected from the group consisting of:
as a further component of constituent (a), a second compound or two or more further compounds of the formula (I) or (ent-I) as defined above in particular in one of the developments which are stated above to be preferred (for example a blend of a first compound of the formula (I), in which R1 to R5 means H and a second compound of the formula (I), in which R1 to R4 mean H and R5 means methyl),
as constituent (b), a compound or a mixture of two, three or more compounds of the formulae (IIa), (IIb), (IIc), (ent-IIa), (ent-IIb), (ent-IIc)

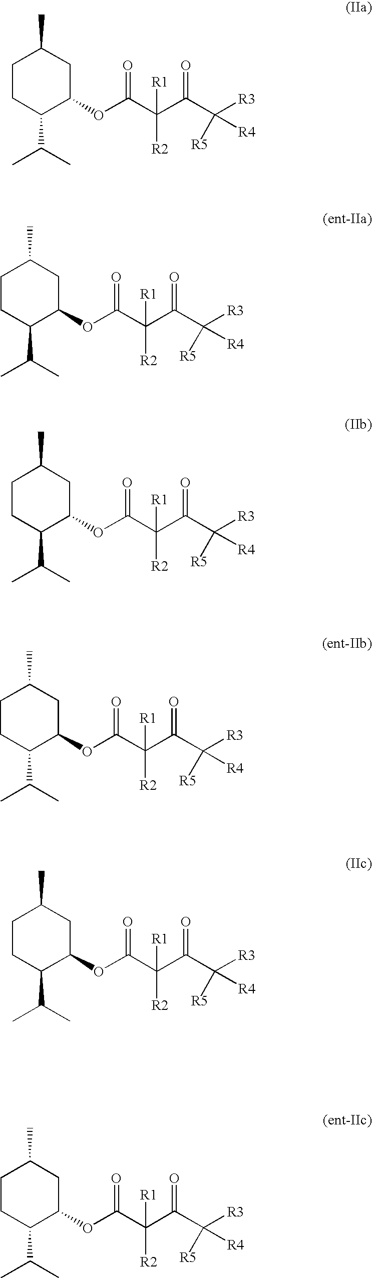

wherein, in each of the formulae (IIa), (IIb), (IIc), (ent-IIa), (ent-IIb) and (ent-IIc), R1, R2, R3, R4 and R5 in each case mutually independently have one of the meanings for the formulae (I) and (ent-I) stated above,
wherein the particular meanings of R1, R2, R3, R4 and R5 for the compounds of the formulae (I), (ent-I), (IIa), (IIb), (IIc), (ent-IIa), (ent-IIb) and (ent-IIc) present in the blend are in each case mutually independent,
as constituent (c), one or more further substances having a physiological cooling action, wherein the further substance or one, several or all of the further substances (i) cause(s) a flavor effect or (ii) cause(s) no flavor effect,
as constituent (d), one or more aroma substances without a physiological cooling action. For the purposes of the present text, aroma substances are preferably substances which are produced from natural primary materials in particular such substances which occur in a natural edible material and have an aromatizing action therein (=natural aroma substances). However, the term “aroma substances” additionally also comprises substances which are added to edible materials for aromatization (=nature-identical and/or artificial aroma substances).
as constituent (e), one or more substances without a physiological cooling action which have a trigeminal or salivatory action,
   wherein, if L-menthyl 3-oxobutyrate is used as the first compound of the formula (I) and a quantity of L-menthyl 3-hydroxybutyrate which causes a physiological cooling action is used as or in constituent (c), the blend does not comprise a reducing agent for L-menthyl 3-oxobutyrate.

A blend according to the invention is thus obtained if, in addition to a first compound of the formula (I) or (ent-I) as defined above, preferably in one of the developments which are stated above to be preferred, a second compound or two or more further compounds of the formula (I) or (ent-I) as defined above are present as a further component.

A blend according to the invention is also obtained if, in addition to a first compound of the formula (I) or (ent-I) as defined above, a compound or a mixture of two, three or more compounds of the formulae (IIa), (IIb), (IIc), (ent-IIa), (ent-IIb) and (ent-IIc) are present as constituent (b). It is frequently the case here for not just one first compound of the formula (I) or (ent-I) to be present in addition to one or more compounds of the formulae (IIa), (IIb), (IIc), (ent-IIa), (ent-IIb) and (ent-IIc), but instead at least two compounds of the formulae (I) or (ent-I).

A blend according to the invention is likewise obtained if, in addition to at least one first compound of the formula (I) or (ent-I), one or more further substances having a physiological cooling action are present as constituent (c).

The same applies in the presence of one or more aroma substances without a physiological cooling action, which may be present as constituent (d) of a blend according to the invention. The same likewise applies if one or more substances having a trigeminal or salivatory action without a physiological cooling action are present as constituent (e). In each case, a blend according to the invention is obtained. Constituents (b), (c), (d) and (e) are preferably present in combination with one another and in addition to constituent (a); preferred combinations are stated below.

If L-menthyl 3-oxobutyrate is used as the first compound of the formula (I) and a quantity of L-menthyl 3-oxobutyrate which causes a physiological cooling action is used as or in constituent (c), a blend according to the invention preferably does not comprise a reducing agent for L-menthyl 3-oxobutyrate. The L-menthyl 3-oxobutyrate is thus not reduced in the blend.

The compounds of the formulae (IIa), (IIb), (IIc), (ent-IIa), (ent-IIb) and (ent-IIc), like the compounds of the formulae (I) and (Ia), fall within the general formula (III):

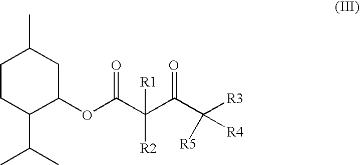

If, in compounds of type (III), R1 and/or R2 is H, the compounds may, at least in part, also be present in the corresponding tautomeric enol forms. This is in particular dependent on the polarity, the pH value and the temperature of the medium in which the compounds have been dissolved or incorporated.

A blend according to the invention preferably comprises a compound or a mixture of compounds of the formulae (IIa), (IIb) and/or (IIc) as or in constituent (b).

Preferred blends according to the invention are those which comprise constituent (a) and preferably (b) (and additionally optionally also constituents (c), (d) and/or (e)), wherein, in one, two, three, more than three or all the compounds (I), (ent-I), (IIa), (IIb), (IIc), (ent-IIa), (ent-IIb) and (ent-IIc) contained in the blend, R1, R2, R3, R4 and R5 have the meanings which are stated above to be preferred for the formulae (I) and (ent-I).

In preferred blends according to the invention, in particular in blends according to the invention which as or in constituent (b) comprise a compound or a mixture of compounds of the formulae (IIa), (IIb) and/or (IIc), and furthermore in particular in blends according to the invention in which all the compounds which fall within the general formula (III) comprise groups R1, R2, R3, R4 and R5 which are stated above to be preferred, the weight ratio of (a) the entirety of compounds of the formula (I) and (ent-I) to (b) the entirety of compounds of the formulae (IIa), (IIb), (IIc), (ent-IIa), (ent-IIb) and (ent-IIc) is in the range from 200:1 to 4:1, preferably in the range from 100:1 to 10:1, particularly preferably in the range from 100:1 to 20:1.

The invention is based on the surprising recognition that the above-stated menthyl 3-oxocarboxylic acid esters of the formulae (I) and (ent-I) and the above-described blends thereof, in particular the compound of the formula (I) and the corresponding blends, cause a strong and long-lasting sensation of coldness on the skin or mucous membranes, in particular on the mucous membranes of the oral, nasal and pharyngeal cavities. Said compounds here exhibit no other trigeminal effects such as spiciness, tingling or numbing and are furthermore not bitter. At the same time, the compounds to be used according to the invention of the formulae (I) and (ent-I) are resistant to hydrolysis within the bounds of conventional water-containing formulations and preparation conditions in the range from pH 1 to pH 12, in particular in the range from pH 4 to pH 9, such that they have an extended storage life in formulations and preparations, so meaning that the particular formulation or preparation itself has a long storage life.

In order to achieve a cooling action which is as strong as possible, in preferred blends according to the invention, i.e. in the case of combinations of two or more compounds of the formulae (I) and/or (ent-I) or in the case of combinations of at least one compound of the formula (I) or (ent-I) with compounds of the formulae (IIa), (IIb), (IIc), (ent-IIa), (ent-IIb) and (ent-IIc), the highest possible proportion of compounds of the formula (I) is selected, i.e. preferably greater than or equal to 90 wt. %, preferably greater than or equal to 95 wt. %, relative to the total weight of all the compounds of the formulae (I), (ent-I), (IIa), (IIb), (IIc), (ent-IIa), (ent-IIb) and (ent-IIc) (compounds of the general formula (III)) contained in the blend. A blend according to the invention thus preferably comprises one or more compounds of the formula (I), wherein the proportion of compounds of the formula (I) to the total weight of compounds of the formulae (I), (ent-I), (IIa), (IIb), (IIc), (ent-IIa), (ent-IIb) and (ent-IIc) amounts to at least 90 wt. %, preferably at least 95 wt. %.

Blends according to the invention which are particularly preferred are those which, in addition to constituent (a) (and optionally constituents (b), (d) or (e)), comprise as constituent (c) one or more further substances having a physiological cooling action, these latter substances causing no flavor effect and no aroma action; the substances having a physiological cooling action thus preferably merely have a (substantially) “pure” cooling action without any further sensory effect. This prevents the aroma profile of the blend being for example shifted towards “mint” (peppermint).

Very particularly preferably blends according to the invention are those consisting of or comprising a constituent (a) (and optionally constituents (b), (c) or (e)) and, as constituent (d), one or more aroma substances without a physiological cooling action, wherein this aroma substance or these aroma substances preferably causes or cause a flavor impression, a flavor-modulating effect, a trigeminal effect and/or a salivatory stimulus. One or more substances which cause other trigeminal stimuli or a salivatory action may, however, also be provided as constituent (e) in addition to or instead of constituent (d). The corresponding blends according to the invention have a pleasant cooling action and balanced sensory profile simultaneously combined with elevated impact, i.e. a strong initial flavor impression.

A blend according to the invention preferably comprises as constituent (c) one, two or more compounds selected from the group consisting of: menthol and menthol derivatives (for example L-menthol, D-menthol, racemic menthol, isomenthol, neoisomenthol, neomenthol), menthyl ethers (for example (I-menthoxy)-1,2-propanediol, (1-menthoxy)-2-methyl-1,2-propanediol, I-menthyl methyl ether), menthyl esters (for example menthyl formate, menthyl acetate, menthyl isobutyrate, menthyl lactate, L-menthyl L-lactate, L-menthyl D-lactate, menthyl (2-methoxy)acetate, menthyl (2-methoxyethoxy)acetate, menthyl pyroglutamate), menthyl carbonates (for example menthyl propylene glycol carbonate, menthyl ethylene glycol carbonate, menthyl glycerol carbonate or mixtures thereof), the semiesters of menthols with a dicarboxylic acid or the derivatives thereof (for example monomenthyl succinate, monomenthyl glutarate, monomenthyl malonate, O-menthyl succinate N,N-(dimethyl)amide, O-menthyl succinamide), menthane carboxamides (for example menthane carboxylic acid N-ethylamide [WS3], N^{α}-(menthanecarbonyl) glycine ethyl ester [WS5], menthane carboxylic acid N-(4-cyanophenyl)amide, menthane carboxylic acid N-(alkoxyalkyl)amides), menthone and menthone derivatives (for example L-menthone glycerol ketal), 2,3-dimethyl-2-(2-propyl)-butanoic acid derivatives (for example 2,3-dimethyl-2-(2-propyl)-butanoic acid N-methylamide [WS23]), isopulegol or the esters thereof (1-(−)-isopulegol, I-(−)-isopulegol acetate), menthane derivatives (for example p-menthane-3,8-diol), cubebol or synthetic or natural blends containing cubebol, pyrrolidone derivatives of cycloalkyldione derivatives (for example 3-methyl-2(1-pyrrolidinyl)-2-cyclopenten-1-one) or tetrahydropyrimidin-2-ones (for example icilin or related compounds, as described in WO 2004/026840).

Menthol (L-menthol, D-menthol, racemic menthol, isomenthol, neoisomenthol, neomenthol), L-menthyl methyl ether, menthyl formate, menthyl acetate), menthone, isopulegol, I-(−)-isopulegol acetate) and cubebol here have flavor effect.

The one or more further substances having a physiological cooling action which may be used as constituent (c) in a blend according to the invention are here preferably selected from the following list: menthol and menthol derivatives (for example L-menthol, D-menthol, racemic menthol, isomenthol, neoisomenthol, neomenthol), menthyl ethers (for example (1-menthoxy)-1,2-propanediol, (1-menthoxy)-2-methyl-1,2-propanediol, 1-menthyl methyl ether), menthyl esters (for example menthyl formate, menthyl acetate, menthyl isobutyrate, menthyl lactates, L-menthyl L-lactate, L-menthyl D-lactate, menthyl (2-methoxy)acetate, menthyl (2-methoxyethoxy)acetate, menthyl pyroglutamate), menthyl carbonates (for example menthyl propylene glycol carbonate, menthyl ethylene glycol carbonate, menthyl glycerol carbonate or mixtures thereof), the semiesters of menthols with a dicarboxylic acid or the derivatives thereof (for example monomenthyl succinate, monomenthyl glutarate, monomenthyl malonate, O-menthyl succinate N,N-(dimethyl)amide, O-menthyl succinamide), menthane carboxamides (for example menthane carboxylic acid N-ethylamide [WS3], N^{α}-(menthanecarbonyl) glycine ethyl ester [WS5], menthane carboxylic acid N-(alkoxyalkyl)amides), menthone and menthone derivatives (for example L-menthone glycerol ketal), 2,3-dimethyl-2-(2-propyl)-butanoic acid derivatives (for example 2,3-dimethyl-2-(2-propyl)-butanoic acid N-methylamide [WS23]), isopulegol or the esters thereof (1-(−)-isopulegol, I-(−)-isopulegol acetate), menthane derivatives (for example p-menthane-3,8-diol), cubebol or synthetic or natural blends containing cubebol, pyrrolidone derivatives of cycloalkyldione derivatives (for example 3-methyl-2(1-pyrrolidinyl)-2-cyclopenten-1-one).

Mixtures of a compound of the formula (I) with R1, R2, R3, R4 and R5=H and L-menthyl lactate surprisingly exhibited a longer lasting cooling action than the individual components; lengthening of the cooling action is probably due to synergistic action.

The one or more further substances having a physiological cooling action which may be used as constituent (c) of a blend according to the invention are preferably substances which at least substantially cause a physiological cooling action without simultaneously causing a flavor action. Such preferred substances are: menthyl ethers (for example (1-menthoxy)-1,2-propanediol, (1-menthoxy)-2-methyl-1,2-propanediol), relatively highly polar menthyl esters (for example menthyl lactate, L-menthyl L-lactate, L-menthyl D-lactate, menthyl (2-methoxy)acetate, menthyl (2-methoxyethoxy)acetate, menthyl pyroglutamate), menthyl carbonates (for example menthyl propylene glycol carbonate, menthyl ethylene glycol carbonate, menthyl glycerol carbonate), the semiesters of menthols with a dicarboxylic acid or the derivatives thereof (for example monomenthyl succinate, monomenthyl glutarate, monomenthyl malonate, O-menthyl succinate N,N-(dimethyl)amide, O-menthyl succinamide), menthane carboxamides (for example menthane carboxylic acid N-ethylamide [WS3], N^{α}-(menthanecarbonyl)glycine ethyl ester [WS5], menthane carboxylic acid N-(4-cyanophenyl)amide, menthane carboxylic acid N-(alkoxyalkyl)amides), menthone derivatives (for example L-menthone glycerol ketal), 2,3-dimethyl-2-(2-propyl)-butanoic acid derivatives, (for example 2,3-dimethyl-2-(2-propyl)-butanoic acid N-methylamide), pyrrolidone derivatives of cycloalkyldione derivatives (for example 3-methyl-2(1-pyrrolidinyl)-2-cyclopenten-1-one) or tetrahydropyrimidin-2-ones (for example icilin or related compounds which are described in WO 2004/026840).

Specific blends of menthane carboxamides with cooling active ingredients such as acyclic carboxamides and L-menthyl lactate and optionally further cooling active ingredients or trigeminal stimulants are described for example in WO 2005/0117811; said document, however, makes no mention of using the menthyl 3-oxocarboxylic acid esters according to the invention (compounds of the formulae (I) and (ent-I)).

The compounds of the formula (I) or the corresponding mixtures are preferably synthesized by reaction of the corresponding 3-oxoalkanecarboxylic acid methyl or ethyl esters or another corresponding activated 3-oxoalkanecarboxylic acid derivative with a corresponding 2-isopropyl-5-methyl-cyclohexanol without solvent or in an aprotic solvent such as acetone, ether, methylene chloride, tetrahydrofuran or toluene in the absence or presence of known Lewis acids such as zinc chloride, tin(IV) chloride, iron(III) chloride or in the presence other transesterification catalysts such as pyridine, imidazole or 4-dimethylaminopyridine (DMAP) (see Eur. J. Org. Chem. 2000, 1633 and the literature cited therein for a review of methods known from the literature for synthesizing 3-oxoalkanoic acid esters).

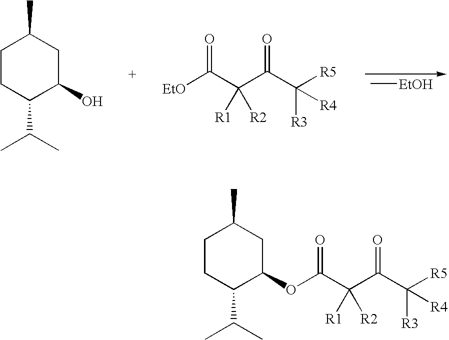

The synthesis of compound (I) with R1 to R5=H has repeatedly been described in the literature (H. Hennecka in E. Müller (ed.) “Houben-Weyl, Methoden der organischen Chemie”, vol. 8, page 527, Georg Thieme Verlag, Stuttgart 1952; and literature cited therein).

Corresponding synthetic pathways are preferred for the production of a compound of the formula (ent-I) and for the production of compounds of the formulae (IIa), (IIb), (IIc), (ent-IIa), (ent-IIb), (ent-IIc).

The crude products of the synthesis are preferably purified or concentrated by physical, optionally also enantioselective or enantiospecific separation methods, for example extraction, partition methods, crystallization, distillation, chromatography, sublimation, steam distillation, reverse osmosis, permeation or the like, the separation method preferably being selected such that, after the separation operation, the menthyl 3-oxocarboxylic acid esters of the formula (I) or (ent-I) or mixtures thereof are present in a proportion of greater than 90 wt. %, preferably greater than 95 wt. %, relative to the total quantity of compounds of the formulae (I), (ent-I), (IIa), (IIb), (tic), (ent-IIa), (ent-IIb) and (ent-IIc) (i.e. compounds of the general formula (III)) present in the purified product.

Preferred blends according to the invention have already been described above, which, in addition to a compound of the formula (I) or (ent-I) or a blend of such compounds (as constituent (a)), comprise as constituent (c) one or more further substances having a physiological cooling action. It has already been explained in this connection that the further substance or one, several or all of the further substances which are used as constituent (c) preferably cause no flavor effect; preferred corresponding further substances having a physiological cooling action have been stated. Particularly preferred blends according to the invention contain 0.05 to 90 wt. % of constituent (a) and 0.01 to 90 wt. % of constituent (c), relative to the total weight of constituents (a), (b), (c), (d) and (e) (if present), wherein the respective explanations stated above with regard to preferred developments of constituents (a) and (c) should be noted. In addition to constituents (a) and (c), such a preferred blend may additionally comprise one or more of constituents (b), (d) or (e), as are stated further above. A preferred blend according to the invention may additionally comprise one or more components from the group consisting of: solvents, carriers, other auxiliary substances (such as for example dyes, preservatives, stabilizers and/or thickeners). It is particularly preferred in blends according to the invention to use one or more aroma substances without a physiological cooling action (constituent (d)), wherein these aroma substances, in addition to their actual odorous aroma value, preferably also cause a flavor impression, a flavor-modulating effect or a trigeminal, but non-cooling effect and/or a salivatory stimulus. If said aroma substances cause a trigeminal or salivatory stimulus, they may simultaneously be regarded as constituent (e) of a blend according to the invention. It is preferred to use as or in constituent (d) aroma substances which cause one or more of the following preferred flavor impressions, flavor-modulating effects or trigeminal stimuli:

preferred flavor impressions: sweet, umami, bitter, salty, sour;
preferred flavor-modulating effects: bitter-masking, umami-enhancing, sweet-enhancing, salt-enhancing, sour-masking;
preferred trigeminal stimuli: spiciness, heat, tingling, pungency.

Pellitorines according to WO 2004/000787 or US 2004/0241312 and alkene carboxylic acid N-alkylamides according to DE 103 51 422 are particularly preferred as or in constituent (d) of a blend according to the invention, i.e. as aroma substances without a physiological cooling action.

The invention also relates to preparations consumed for nutrition or pleasure or used for oral hygiene or pharmaceutical or cosmetic preparations comprising a quantity of a compound of the formula (I) or (ent-I) or a blend consisting of two, three or more compounds of the formula (I) or (ent-I) as defined above, in particular in a development which is stated to be preferred, which is sufficient to achieve a physiological cooling action on the skin and/or mucous membranes. In particular, the quantity used of the compound or blend according to the invention should be sufficient to achieve a physiological cooling action on the mucous membranes in the oral, nasal and/or pharyngeal cavities.

A preparation according to the invention comprises or preferably consists of a blend according to the invention. If, in this blend, L-menthyl 3-oxobutyrate is used as the first compound of the formula (I) and a quantity of L-menthyl 3-hydroxybutyrate which causes a physiological cooling action is used as or in constituent (c), the preparation preferably does not comprise a reducing agent for L-menthyl 3-oxobutyrate. The preparation also preferably comprises no constituents which are capable of converting L-menthyl 3-oxobutyrate by (bio)chemical reactions into L-menthyl 3-hydroxybutyrate.

Preferred preparations according to the invention comprise conventional basic materials, auxiliary substances and additives for preparations consumed for nutrition or for pleasure or used for oral hygiene or for pharmaceutical or cosmetic preparations. A preferred preparation according to the invention comprises
a total of 0.0001 wt. % to 20 wt. %, preferably 0.0001 to 10 wt. %, particularly preferably 0.001 wt. % to 0.5 wt. % of compounds of the formula (I), relative to the total weight of the preparation,
a total of 0.0000001 to 99.99 wt. %, preferably 10 to 80 wt. % of compounds of the formula (ent-I), constituents (b), (c), (d) and/or (e) as defined above, in particular in a development which is stated to be preferred, and further basic materials, auxiliary substances and additives with the exception of water, relative to the total weight of the preparation,
0 to 99.99 wt. % of water, relative to the total weight of the preparation, preferably 5 to 80 wt. %.

Preferred preparations consumed for nutrition or for pleasure are for example bakery products (for example bread, dry biscuits, cakes, other pastry products), confectionery (for example chocolates, chocolate bar products, other bar products, fruit gums, hard and soft caramels, chewing gum), alcoholic or non-alcoholic beverages (for example coffee, tea, wine, beverages containing wine, beer, beverages containing beer, liqueurs, spirits, brandies, fruit-containing carbonated beverages, isotonic beverages, soft drinks, nectars, fruit and vegetable juices, fruit or vegetable juice preparations), instant beverages (for example instant cocoa beverages, instant tea beverages, instant coffee beverages), meat products (for example ham, fresh or cured sausage preparations, spiced or marinated fresh or cured meat products), eggs or egg products (dried egg, egg white, egg yolk), cereal products (for example breakfast cereals, muesli bars, precooked ready rice products), dairy products (for example milk beverages, milk ice cream, yogurt, kefir, curd cheese, soft cheese, hard cheese, dried milk powder, whey, butter, buttermilk), fruit preparations (for example jams, fruit ice cream, fruit sauces, fruit fillings), vegetable preparations (for example ketchup, sauces, dried vegetables, deep-frozen vegetables, precooked vegetables, preserved vegetables), snack articles (for example baked or fried potato chips or potato dough products, maize- or peanut-based extrudates), fat- or oil-based products or emulsions thereof (for example mayonnaise, remoulade, dressings), other ready-to-serve meals and soups (for example dried soups, instant soups, precooked soups), spices, seasoning mixtures and in particular powdered seasonings, which are for example used in snack food applications. The preparations for the purposes of the invention may also be used as semifinished products for the production of further preparations consumed for nutrition or for pleasure. The preparations for the purposes of the invention may also be nutritional supplements in the form of capsules, tablets (uncoated and coated tablets, for example coatings resistant to gastric juices), sugar-coated tablets, granules, pellets, mixtures of solids, dispersions in liquid phases, as emulsions, as powders, as solutions, as pastes or as other swallowable or chewable preparations.

Preparations used for oral hygiene are preferably toothpaste, tooth cream, tooth gel, tooth powder, dental cleaning liquid, dental cleaning foam, mouthwash, tooth cream and mouthwash as a 2-in-1 product, hard candies, mouth spray, dental floss or dental care chewing gum.

Dental care products (as the basis for preparations for oral care purposes) which contain the compounds, mixtures or blends according to the invention generally comprise an abrasive system (abrasive or polishing agent), such as for example silicas, calcium carbonates, calcium phosphates, aluminum oxides and/or hydroxyapatites, surface-active substances such as for example sodium lauryl sulfate, sodium lauryl sarcosinate and/or cocamidopropyl betaine, humectants such as for example glycerol and/or sorbitol, thickeners, such as for example carboxymethylcellulose, polyethylene glycols, carrageenan and/or Laponite®, sweeteners, such as for example saccharin, sodium cyclamate, sucralose, acesulfame-K or sugar alcohol flavor-correcting agents for unpleasant flavor impressions such as for example hydroxyflavanones according to US 2002/0188019, flavor-correcting agents for further, generally not unpleasant flavor impressions, flavor-modulating substances (for example inositol phosphate, nucleotides such as guanosine monophosphate, adenosine monophosphate or other substances such as sodium glutamate or 2-phenoxypropionic acid), cooling active ingredients such as for example menthol, menthol derivatives (for example L-menthol, L-menthyl lactate, L-menthyl alkylcarbonates, menthone ketals, menthane carboxamides), 2,2,2-trialkylacetamides (for example 2,2-diisopropyl propionic acid methylamide), icilin and icilin derivatives, stabilizers and active ingredients, such as for example sodium fluoride, sodium monofluorophosphate, tin difluoride, quaternary ammonium fluorides, zinc citrate, zinc sulfate, tin pyrophosphate, tin dichloride, blends of different pyrophosphates, triclosan, cetylpyridinium chloride, aluminum lactate, potassium citrate, potassium nitrate, potassium chloride, strontium chloride, hydrogen peroxide, aromas and/or sodium bicarbonate or flavor-correcting agents.

Chewing gums (as a further example of the preparations for oral care purposes) which contain the compounds, mixtures or blends according to the invention generally comprise a chewing gum base, i.e. a chewable mass which becomes plastic on chewing, sugars of various kinds, sugar substitutes, other sweet-tasting substances, sugar alcohols, flavor-correcting agents for unpleasant flavor impressions, other flavor modulators for further, generally not unpleasant flavor impressions, flavor-modulating substances (for example inositol phosphate, nucleotides such as guanosine monophosphate, adenosine monophosphate or other substances such as sodium glutamate or 2-phenoxypropionic acid), humectants, thickeners, emulsifiers, aromas and stabilizers or flavor-correcting agents.

Pharmaceutical preparations according to the invention which are preferred for the purposes of the invention are oral preparations, which for example assume the form of capsules, tablets (uncoated and coated tablets, for example coatings resistant to gastric juices), sugar-coated tablets, granules, pellets, mixtures of solids, dispersions in liquid phases, emulsions, powders, solutions, pastes or other swallowable or chewable preparations and are used as prescription-only, drugstore-only or other medicaments or as nutritional supplements.

Cosmetic preparations according to the invention may for example be present in one of the following forms: soap, synthetic detergent, a liquid washing, shower or bath preparation, emulsion (as a solution, dispersion, suspension, cream, lotion or milk depending on the production method and constituents of the “water-in-oil” (W/O), “oil-in-water” (O/W) or multiple emulsion, PIT emulsion, emulsion foam, microemulsion, nanoemulsion or Pickering emulsion type), ointment, paste, gel (including hydrogel, hydrodispersion gel, oleogel), oil, toner, balsam, serum, powder, eau de toilette, toilet water, eau de cologne, perfume, wax, as a stick, roll-on, (pump) spray, aerosol (foaming, non-foaming or post-foaming), as a foot care product (including keratolytics, deodorant), beard shampoo or care preparations, insect-repellent product, sunscreen product, aftersun preparation, shaving preparation (for example shaving foams, soaps or gels) or aftershave preparation (balm, lotion), depilatory product, hair care product such as for example shampoo (including 2-in-1 shampoo, antidandruff shampoo, baby shampoo, shampoo for a dry scalp, shampoo concentrate), conditioner, hair tonic, hair water, hair rinse, hairdressing cream, pomade, permanent wave and setting lotion, hair smoothing product (detangling product, relaxer), hair strengthener, styling aid (for example gel or wax), blending product, hair lightener, hair conditioner, hair mousse, hair toning product, hair dyes (for example temporary, substantive, semipermanent, permanent hair dyes), nail care products such as for example nail polish and nail polish remover, deodorant and/or antiperspirant, mouthwash, water pick, makeup, makeup remover, eye care preparation, lip cosmetics, lip care preparation, decorative cosmetics (for example powder, eye shadows, kohl pencil, lipstick), bath articles (for example capsules) or mask.

Preparations according to the invention which comprise compounds to be used according to the invention or a blend according to the invention are preferably produced by incorporating the compound, the mixture or the blend, for example a blend comprising a solid or liquid carrier in addition to a compound according to the invention, into a base preparation. Advantageously, blends according to the invention, which are initially in solution form and comprise a compound to be used according to the invention, are converted into a solid preparation by spray drying.

According to an alternative, preferred embodiment, preparations according to the invention may be produced by initially incorporating the compounds or blends to be used according to the invention, optionally with further constituents of the preparation according to the invention, into emulsions, into liposomes, for example starting from phosphatidyl choline, into microspheres, into nanospheres or also into capsules, granules or extrudates prepared from a matrix suitable for foodstuffs and products consumed for pleasure, for example prepared from starch, starch derivatives (for example modified starch), cellulose or cellulose derivatives (for example hydroxypropylcellulose), other polysaccharides (for example dextrin, alginate, curdlan, carageenan, chitin, chitosan, pullulan), natural fats, natural waxes (for example beeswax, carnauba wax), prepared from proteins, for example gelatin or other natural products (for example shellac) or non-natural matrix materials (such as polyurea). In said embodiment, depending on the matrix, the products may be treated by spray drying, spray granulation, melt granulation, coacervation, coagulation, extrusion, melt extrusion, emulsion methods, coating or other suitable encapsulation methods and optionally a suitable combination of the above-stated methods.

In a further preferred production method, the compounds to be used according to the invention or blends according to the invention are initially complexed with one or more suitable complexing agents, for example with cyclodextrins or cyclodextrin derivatives, preferably alpha-, beta- or gamma-cyclodextrin, and in used in this complexed form.

A particularly preferred preparation according to the invention is one in which the matrix is selected such that the compounds to be used according to the invention or blends according to the invention, in particular blends comprising further cooling active ingredients and/or aromas, are released from the matrix in delayed manner, such that a long-lasting cooling action is achieved.

Constituents for preparations consumed for nutrition or for pleasure according to the invention which may be used are conventional basic materials, auxiliary substances and additives for foodstuffs or products consumed for pleasure, for example water, mixtures of fresh or processed, plant or animal basic or raw materials (for example raw, roasted, dried, fermented, smoked and/or boiled meat, bone, cartilage, fish, vegetables, fruit, herbs, nuts, vegetable or fruit juices or pastes or mixtures thereof), digestible or non-digestible carbohydrates (for example sucrose, maltose, fructose, glucose, dextrins, amylose, amylopectin, inulin, xylans, cellulose, tagatose), sugar alcohols (for example sorbitol, erythritol), natural or hardened fats (for example tallow, lard, palm fat, coconut oil, hardened vegetable fat), oils (for example sunflower oil, peanut oil, maize germ oil, olive oil, fish oil, soya oil, sesame oil), fatty acids or the salts thereof (for example potassium stearate), proteinogenic or non-proteinogenic amino acids and related compounds (for example γ-aminobutyric acid, taurine), peptides (for example glutathione), native or processed proteins (for example gelatin), enzymes (for example peptidases), nucleic acids, nucleotides, flavor-correcting agents for unpleasant flavor impressions, further flavor-modulators for further generally not unpleasant flavor impressions, other flavor-modulating substances (for example inositol phosphate, nucleotides such as guanosine monophosphate, adenosine monophosphate or other substances such as sodium glutamate or 2-phenoxypropionic acid), emulsifiers (for example lecithins, diacylglycerols, gum arabic), stabilizers (for example carrageenan, alginate), preservatives (for example benzoic acid, sorbic acid), antioxidants (for example tocopherol, ascorbic acid), chelating agents (for example citric acid), organic or inorganic acidulants (for example malic acid, acetic acid, citric acid, tartaric acid, phosphoric acid), bitter substances (for example quinine, caffeine, limonene, amarogentin, humolone, lupolone, catechins, tannins), mineral salts (for example sodium chloride, potassium chloride, magnesium chloride, sodium phosphates), substances preventing enzymatic browning (for example sulfite, ascorbic acid), essential oils, plant extracts, natural or synthetic dyes or coloring pigments (for example carotenoids, flavonoids, anthocyans, chlorophyll and the derivatives thereof), spices, trigeminally active substances or plant extracts containing such trigeminally active substances, synthetic, natural or nature-identical aroma substances or odoriferous substances and flavor-correcting agents.

A further aspect of the present invention relates to a method for achieving a physiological cooling action on the skin and/or mucous membranes. Such a method according to the invention may be carried out for therapeutic or non-therapeutic (for example cosmetic) purposes and comprises the following step:
application of a quantity sufficient to achieve a physiological cooling action
   (i) of a compound of the formula (I) or (ent-I) or a blend consisting of two, three or more compounds of the formula (I) or (ent-I) (as defined above, preferably in an above-described preferred development),
   (ii) of a blend according to the invention (as described above, preferably in a development described as being preferred) or
   (iii) of a preparation according to the invention (as described above, preferably in a development which is stated to be preferred), onto the skin and/or mucous membranes.

Further aspects of the present invention emerge from the following Examples and the appended claims.

### EXAMPLES

The Examples merely serve to illustrate the invention without thereby limiting it. Unless otherwise stated, all stated values relate to weight.

### Example 1

### Synthesis of (L)-menthyl 3-oxo-butyrate (I; R1 to R5=H)

A mixture of 156 g (1 mol) of (L)-menthol and 130 g (1 mol) of ethyl acetoacetate was heated with stirring to a temperature of 140° C. and the ethanol liberated on onset of the reaction was removed by distillation through a 15 cm Vigreux column with attached condenser. Once removal of the ethanol by distillation was complete (approx. 5 h), the residue remaining the reaction flask was subjected to fractional distillation through the connected Vigreux column. At 140-145° C./10 mbar, 235 g (98% of theoretical) of (L)-menthyl 3-oxo-butyrate were obtained as a colorless oil which, after extended standing at room temperature, solidified to yield colorless crystals with an m.p. of 35-36° C.

### Example 2

### Synthesis of (L)-menthyl 3-oxo-pentanoate (I; R1 to R4=H; R5=CH₃)

A mixture of 156 g (1 mol) of (L)-menthol and 142 g (1 mol) of 3-oxopentanoic acid ethyl ester was heated with stirring to a temperature of 140° C. and the ethanol liberated on onset of the reaction was removed by distillation through a 15 cm Vigreux column with attached condenser. Once removal of the ethanol by distillation was complete (approx. 5 h), the residue remaining the reaction flask was subjected to fractional distillation through the connected Vigreux column. At 145-152° C./10 mbar, 248 g (98% of theoretical) of (L)-menthyl 3-oxo-pentanoate were obtained as a colorless oil.

¹H-NMR (CDCl3): δ=0.77 (d, j=6.9 Hz, 3H, H-9), 0.89 (d, j=7.0 Hz, 3H, H-10), 0.91 (d, j=6.6 Hz, 3H, H-7), 0.99 (m, 2H, H-6), 1.09 (t, j=7.3 Hz, 3H, H-13), 1.38 (m, 2H, H-2), 1.49 (m, 1H, H-1), 1.68 (m, 2H, H-5), 1.87 (m, 1H, H-8), 2.03 (m, 1H, H-4), 2.56 (q, j=7.3 Hz, 2H, H-12), 3.42 (s, 2H, H-11), 4.73 ppm (m, 1H, H-3).

Tetramethylsilane (TMS) was used as internal standard.

### Example of Application 1: Cooling Action

The compounds from Examples 1 and 2 were tested for their sensory properties, in particular their cooling action. To this end, they were dissolved, in each case in a specific final concentration, in a mass prepared from sucrose (saccharose) and water (confectioner's fondant, supplier Nordzucker AG, Nordstemmen) and evaluated by a panel of experts. Sensory impressions were rated and the cooling action was assessed on a scale from 1 (no cooling action) to 9 (extremely strong cooling action).

Profile of L-menthyl 3-oxobutyrate (Example 1) at a concentration of 0.05 wt. %, relative to the complete preparation: slightly flowery, cooling action 5-6, not bitter.

Profile of L-menthyl 3-oxopentanoate (Example 2) at a concentration of 0.05 wt. %, relative to the complete preparation: cooling action 5-6; slower onset but longer lasting than Example 1, not bitter. With regard to skin-cooling characteristics, it was found (application site: forearm of human test subjects) that the compounds according to the invention, in particular L-menthyl 3-oxobutyrate (I-X) and L-menthyl 3-oxopentanoate (I-XX), exhibit a similar cooling profile over time as the frequently used cooling active ingredient L-menthyl lactate (Frescolat ML®, Symrise GmbH & Co. KG), with an overall weaker intensity of cooling being observed than for this comparison substance.

### Example of Application 2: Aroma Blend for Achieving a Cooling Action

| | Proportion |
|---|---|
| Constituent | in % |
| (L)-menthyl 3-oxobutyrate (Example 1) | 25 |
| L-Menthyl lactate (Frescolat ML, Symrise) | 65 |
| O-L-Menthyl-O′-(2-hydroxyethyl) carbonate (Frescolat | 10 |
| MGC, Symrise) | |

A strongly cooling, but otherwise virtually flavorless and odorless aroma blend which is liquid at room temperature (20° C.) is obtained by blending the components.

### Example of Application 3: Aroma Blend for Achieving a Cooling Action

| | Proportion |
|---|---|
| Constituent | in % |
| (L)-menthyl 3-oxopentanoate (Example 2) | 7.5 |
| L-Menthane carboxylic acid N-ethylamide (WS 3, for | 5 |
| example Millennium) | |
| L-Menthyl lactate (Frescolat ML, Symrise) | 32.5 |
| O-L-Menthyl-O′-(2-hydroxyethyl) carbonate (Frescolat | 5 |
| MGC, Symrise) | |
| Propylene glycol | 50 |

A strongly cooling, but otherwise virtually flavorless and odorless aroma blend which is liquid at room temperature (20° C.) is obtained by blending the components.

### Example of Application 4: Aroma Blend for Achieving an Aromatizing and Cooling Action

| | Proportion |
|---|---|
| Constituent | in % |
| (L)-menthyl 3-oxobutyrate (Example 1) | 15 |
| Peppermint oil | 10 |
| L-Menthyl lactate (Frescolat ML, Symrise) | 65 |
| O-L-Menthyl-O′-(2-hydroxyethyl) carbonate (Frescolat | 10 |
| MGC, Symrise) | |

A strongly cooling aroma blend with a strong odor of peppermint is obtained by blending the components

Example of Application 5: Aroma Blend for Achieving a Cooling Action with a Simultaneous Tingling Effect

| | Proportion |
|---|---|
| Constituent | in % |
| (L)-menthyl 3-oxobutyrate (Example 1) | 15 |
| Solution of 10 wt. % pellitorine in propylene | 10 |
| glycol/peppermint oil | |
| L-Menthyl lactate (Frescolat ML, Symrise) | 65 |
| O-L-Menthyl-O′-(2-hydroxyethyl) carbonate (Frescolat | 10 |
| MGC, Symrise) | |

A strongly cooling aroma blend which stimulates salivation and causes a tingling effect is obtained by blending the components.

### Example of Application 6: Use in the Form of an Aroma Blend in a Toothpaste

| | | Quantity used in |
|---|---|---|
| Part | Constituent | wt. % |
| A | Demineralized water | 22.00 |
| | Sorbitol (70%) | 45.00 |
| | Solbrol ® M, sodium salt | 0.15 |
| | (Bayer AG, p-hydroxybenzoic acid alkyl ester) | |
| | Trisodium phosphate | 0.10 |
| | Saccharin, 450x | 0.20 |
| | Sodium monofluorophosphate | 1.12 |
| | Polyethylene glycol 1500 | 5.00 |
| B | Sident 9 (abrasive silicon dioxide) | 10.00 |
| | Sident 22 S (thickening silicon dioxide) | 8.00 |
| | Sodium carboxymethylcellulose | 0.90 |
| | Titanium dioxide | 0.50 |
| C | Demineralized water | 4.53 |
| | Sodium lauryl sulfate | 1.50 |
| D | Aroma blend from Example of application 2 | 1 |

The constituents of parts A and B were in each case individually premixed and in each case thoroughly stirred under a vacuum at 25-30° C. for 30 minutes. Part C was premixed and added to A and B; D was added and the blend was thoroughly stirred under a vacuum at 25-30° C. for a further 30 minutes. After relieving the vacuum, the toothpaste was ready and could be packaged.

### Example of Application 7: Use as Cooling Active Ingredient in a Sugar-Free Chewing Gum

| | | Quantity used in |
|---|---|---|
| Part | Constituent | wt. % |
| A | Chewing gum base, company “Jagum T” | 30.00 |
| B | Powdered sorbitol | 39.00 |
| | Isomalt ® (Palatinit GmbH) | 9.50 |
| | Xylitol | 2.00 |
| | Mannitol | 3.00 |
| | Aspartame ® | 0.10 |
| | Acesulfame ® K | 0.10 |
| | Emulgum ® (Colloides Naturels, Inc.) | 0.30 |
| C | Sorbitol, 70% | 14.00 |
| | Glycerol | 1.00 |
| D | Spearmint/peppermint/eucalyptus | 1 |
| | aroma, containing 5 wt. % | |
| | (L)-menthyl 3-oxobutyrate (Example 1) | |

Parts A to D were mixed and vigorously kneaded. The crude mixture was processed into ready-to-use chewing gum, for example in the form of thin strips.

### Example of Application 8: Use as Cooling Active Ingredient in a Mouthwash

| | | Quantity |
|---|---|---|
| | | used in |
| Part | Constituent | wt. % |
| A | Ethanol | 10.00 |
| | Cremophor ® CO 40 (BASF, detergent) | 1.00 |
| | Benzoic acid | 0.12 |
| | Peppermint/lemon balm aroma containing 0.4 wt. % | 0.25 |
| | pellitorine and 10 wt. % (L)-menthyl 3-oxobutyrate | |
| | (Example 1) | |
| B | Demineralized water | 83.46 |
| | Sorbitol, 70% | 5.00 |
| | Sodium saccharin 450 | 0.07 |
| | L-Blue 5000 e.c., 1% in water (dye) | 0.10 |

The constituents of parts A and B were in each case individually mixed. Part B was slowly stirred into part A until the blend was homogeneous

Example of Application 9: Throat Candies with Liquid/Viscous Core Filling (Centre-Filled Hard Candy)

| | I (wt. %) | II (wt. %) |
|---|---|---|
| Blend A (shell) (80% of the candies) | | |
| Sugar (sucrose) | 58.12 | 49.37 |
| Glucose syrup (solids content 80%) | 41.51 | 49.37 |
| Aroma blend from Example of application 5 | 0.17 | 0.25 |
| I-Menthol | 0.10 | — |
| Lemon oil | 0.10 | 0.10 |
| Citric acid | — | 0.91 |
| Total: | 100 | 100 |
| Blend B (core) (20% of the candies) | | |
| High fructose maize syrup (sugar solids | 84.38 | 84.36 |
| content 85%, only 15% water) | | |
| Glycerol | 15.0 | 15.0 |
| Lecithin | 0.02 | 0.02 |
| Cinnamon oil | — | 0.32 |
| Spearmint oil | 0.28 | — |
| Capsaicin | 0.05 | — |
| Vanillyl alcohol n-butyl ether | — | 0.10 |
| Red dye, as 5% aqueous solution | 0.20 | 0.20 |
| Vanillin | 0.07 | — |
| Total | 100 | 100 |

Candies with a liquid/viscous core were produced on the basis of the methods described in U.S. Pat. No. 6,432,441 (Example 1 therein) and those described in U.S. Pat. No. 5,458,894 or U.S. Pat. No. 5,002,791. The two blends A and B were separately processed to form bases for the shell (blend A) or core (blend B). When consumed by affected individuals, the filled throat candies obtained by means of coextrusion were effective against coughing, sore throat and hoarseness.

### Example of Application 10: Chewing Gum

Chewing gum base K2 consisted of the following ingredients: 28.5% terpene resin, 33.9% polyvinyl acetate (MW=14,000), 16.25% hydrogenated vegetable oil, 5.5% mono- and diglycerides, 0.5% polyisobutene (MW 75,000), 2.0% butyl rubber (isobutene/isoprene copolymer), 4.6% amorphous silicon dioxide (water content approx. 2.5%), 0.05% antioxidant tert.-butylhydroxytoluene (BHT), 0.2% lecithin, and 8.5% calcium carbonate. Chewing gum base K2 and the chewing gum were produced in a similar manner to U.S. Pat. No. 6,986,907.

| | I (wt. %) | II (wt. %) | III (wt. %) |
|---|---|---|---|
| Chewing gum base K2 | 25.30 | 27.30 | 26.30 |
| Sorbitol | 61.48 | 59.48 | 61.80 |
| Glycerol | 2.40 | 2.40 | 2.40 |
| Lecithin | 7.00 | 7.00 | 7.00 |
| Aspartame | 0.14 | 0.14 | 0.14 |
| Encapsulated as partame | 0.68 | 0.68 | 0.68 |
| Menthol, spray-dried | 0.50 | — | — |
| Cherry aroma, spray-dried | — | 1.20 | — |
| Aroma blend from | 1.50 | 1.80 | — |
| Example of application 4, | | | |
| spray-dried | | | |
| Aroma blend from | 1.00 | — | 1.68 |
| Example of application 3 | | | |

The chewing gums of formulations (I) and (II) were shaped into strips, the chewing gum of formulation (III) was shaped into pellets.

### Example of Application 11: Gelatin Capsules for Direct Consumption

| | I (wt. %) | II (wt. %) | III (wt. %) |
|---|---|---|---|
| Gelatin shell: | | | |
| Glycerol | 2.014 | 2.014 | 2.014 |
| Gelatin 240 Bloom | 7.91 | 7.91 | 7.91 |
| Sucralose | 0.065 | 0.065 | 0.065 |
| Allura Red | 0.006 | 0.006 | 0.006 |
| Brilliant blue | 0.005 | 0.005 | 0.005 |
| Core composition: | | | |
| Vegetable oil triglyceride | 79.39 | 68.40 | 58.25 |
| (coconut oil fraction) | | | |
| Cinnamon/aniseed aroma | 10.00 | 20.90 | — |
| Eucalyptus aroma | — | — | 29.95 |
| Neotame and aspartame | 0.01 | 0.05 | — |
| Sucralose | 0.22 | 0.30 | 0.70 |
| Aroma blend from Example of | 0.33 | — | — |
| application 5 | | | |
| Aroma blend from Example of | — | 0.20 | 0.60 |
| application 3 | | | |
| (L)-menthyl 3-oxobutyrate | — | 0.05 | — |
| (Example 1) | | | |
| (−)-Menthone glycerol acetal | — | 0.10 | 0.40 |
| (Frescolat MGA) | | | |
| Vanillin | 0.05 | — | 0.10 |

Gelatin capsules I, II, III suitable for direct consumption were produced according to WO 2004/050069 and in each case had a diameter of 5 mm, the weight ratio of core material to shell material being 90:10. The capsules in each case opened in the mouth within less than 10 seconds and dissolved completely within less than 50 seconds.

### Example of Application 12: Chewable Candy

| | | |
|---|---|---|
| Water | | 7.80% |
| Sugar | Confectioner's sugar C4 | 42.10% |
| Glucose syrup | Dextrose 40 | 37.30% |
| Hardened vegetable fat | Melting point 32-36° C. | 6.60% |
| Lecithin | Emulsifier (soya lecithin) | 0.30% |
| Gelatin | Pig gelatin | 0.80% |
| Fondant | Type - S30 | 4.80% |
| Raspberry aroma | | 0.22% |
| Aroma blend from Example of | | 0.08% |
| application 3 | | |

Manufacturing instructions:
a) allow gelatin to swell in water (1.8 times the quantity of gelatin) at 70° C. for 2 hours;
b) boil sugar, syrup, water, fat and lecithin at 123° C.;
c) slowly mix gelatin solution with the boiled batch;
d) stir in aroma from Example 2 and optionally color;
e) leave the resultant mass to adjust to approx. 70° C. on a cooling table, then add fondant and aerate for approx. 3 minutes on a pulling machine;
f) then chop and package the chewable candy mass.

When the chewable candy is consumed, a fresh, cooling raspberry flavor is perceived during chewing.

### Example of Application 13: Extrudate

| | | |
|---|---|---|
| Glucose syrup, spray-dried | Glucidex IT33W (from | 62.0% |
| (DE value: 31-34) | Roquette) | |
| Maltodextrin (DE value: 17-20) | (from Cerestar) | 28.4% |
| Monomuls emulsifier | Emulsifier based on | 1.8% |
| | hardened palm oil; | |
| | melting point: 64° C. | |
| | (from Grünau) | |
| Dextrose monohydrate (DE value: 99.5) | Dextrose, containing | 1.8% |
| | water of crystallization | |
| | (from Cerestar) | |
| Water | | 2.0% |
| Orange/vanilla aroma | | 3.2% |
| Aroma blend from Example | | 0.8% |
| of application 4 | | |

Manufacturing instructions (see also WO 03/092412):

All constituents were mixed and conveyed into an extruder by single point apportionment. Extrusion temperatures were between 100 and 120° C., specific energy input being 0.2 kWh/kg. The strands emerging from the die plate, which is provided with 1 mm holes, were chopped by rotating blades into approx. 1 mm diameter particles immediately on leaving the die.

### Example of Application 14: Fluidized Bed Granules

A solution consisting of 44 wt. % water, 8 wt. % lemon aroma, 3 wt. % aroma blend from Example of application 4, 13 wt. % gum arabic and 32 wt. % hydrolyzed starch (Maltodextrin DE 15-19) and a little green dye is granulated in a granulating apparatus of the type presented in EP 163 836 (with the following features: diameter of distributor base plate: 225 mm, spray nozzle: two-fluid nozzle; pneumatic classifying discharge: zig-zag pneumatic classifier; filter, internal bag filter). The solution is sprayed into the fluidized bed granulator at a temperature of 32° C. The bed contents are fluidized by blowing in nitrogen in a quantity of 140 kg/h. The inlet temperature of the fluidizing gas is 140° C. The temperature of the exhaust gas is 76° C. The pneumatic classifying gas used is likewise nitrogen in an amount of 15 kg/h with a temperature of 50° C. The contents of the fluidized bed amounts to approx. 500 g. Granulation output amounts to approx. 2.5 kg per hour. Free-flowing granules are obtained having an average particle diameter of 360 micrometers. The granules are round and exhibit a smooth surface. On the basis of the constant pressure drop of the filter and of the likewise constant bed contents, steady-state conditions may be assumed to prevail with regard to the granulation process.

Example of Application 15: Tea Bag with Rooibos or Black Tea and Extrudates from Example of Application 13 or Granules from Example of Application 14

800 g portions of red bush tea (rooibos tea) were mixed in one case with 33 g of the extrudates from Example of application 13 and in one case with 30 g of granules from Example of application 14, portioned and then packaged in tea bags.

800 g portions of black tea (leaf grade: fannings) were mixed in one case with 33 g of the extrudates from Example of application 13 and in one case with 30 g of granules from Example of application 14, portioned and then packaged in tea bags.

### Examples 16-22

### Cosmetic Formulations

16=O/W day cream
17=O/W skin lotion with plan extract
18=after-sun balm
19=body spray for sensitive skin
20=sunscreen lotion (O/W) with broad spectrum protection
21=W/O night cream
22=shampoo

| MATERIAL | | WT. % | | | | | | |
|---|---|---|---|---|---|---|---|---|
| (SUPPLIER) | INCI | 16 | 17 | 18 | 19 | 20 | 21 | 22 |
| -(-alpha-)-Bisabolol, | Bisabolol | | | 0.1 | | | 0.1 | |
| natural (Symrise) | | | | | | | | |
| Abil 350 (Degussa- | Dimethicone | 0.5 | 2.0 | 1.0 | | | | |
| Goldschmidt) | | | | | | | | |
| Allantoin (Merck) | Allantoin | | | 0.1 | | | | |
| Aloe vera gel | Water (aqua), Aloe | | | 3.0 | | | 3.0 | |
| concentrate 10/1 | barbadensis leaf juice | | | | | | | |
| (Symrise) | | | | | | | | |
| Alugel 34 TH | Aluminium stearate | | | | | | 1.0 | |
| (Baerlocher) | | | | | | | | |
| Symatrix (Symrise) | Maltodextrin, Rubus | 0.3 | 0.1 | 1.0 | 0.1 | 0.3 | | |
| | fructicosus (blackberry) | | | | | | | |
| | leaf extract | | | | | | | |
| Butylene glycol | Butylene glycol | | | 5.0 | | | | |
| Carbopol ETD 2050 | Carbomer | | | | | 0.2 | | |
| (Noveon) | | | | | | | | |
| Carbopol Ultrez-10 | Carbomer | | 0.1 | | | | | |
| (Noveon) | | | | | | | | |
| Cetiol OE (Cognis) | Dicaprylyl ether | | | 4.0 | | | | |
| Cetiol SB 45 (Cognis) | Butyrospermum parkii | | | 1.0 | | | | |
| | (shea butter) | | | | | | | |
| Citric acid, 10% solution | Citric acid | | | | | | | 0.3 |
| Comperlan 100 | Cocamide MEA | | | | | | | 0.5 |
| (Cognis) | | | | | | | | |
| Dihydroavenanthramide | | 0.05 | 0.05 | 0.05 | 0.05 | 0.05 | 0.05 | 0.05 |
| D (Symrise) | | | | | | | | |
| Dow Corning 246 Fluid | Cyclohexasiloxane (and) | | | | | 2.0 | | |
| (Dow Corning) | cyclopentasiloxane | | | | | | | |
| Dow Corning 345 Fluid | Cyclomethicone | | | | 0.5 | | | |
| (Dow Corning) | | | | | | | | |
| D-Panthenol (BASF) | Panthenol | | | 1.0 | | | | |
| Dracorin CE (Symrise) | Glyceryl stearate citrate | 5.0 | | | | | | |
| Dracorin GMS | Glyceryl stearate | | 2.0 | | | | | |
| (Symrise) | | | | | | | | |
| Dracorin GOC | Glyceryl oleate citrate, | | | | 2.0 | | | |
| (Symrise) | caprylic/capric triglyceride | | | | | | | |
| Drago-Beta-Glucan | Water (aqua), butylene | 0.3 | | | | | | |
| (Symrise) | glycol, glycerol, Avena | | | | | | | |
| | sativa (oat) kernel extract | | | | | | | |
| Dragocid Liquid | Phenoxyethanol, methyl- | | 0.80 | 0.70 | | 0.70 | 0.80 | |
| (Symrise) | paraben, ethylparaben, | | | | | | | |
| | butylparaben, propyl- | | | | | | | |
| | paraben, isobutylparaben | | | | | | | |
| Dragoderm (Symrise) | Glycerol, Triticum vulgare | | | | | | | 2.0 |
| | (wheat) gluten, water | | | | | | | |
| | (aqua) | | | | | | | |
| Drago-Oat-Active | Water (aqua), butylene | | | | 1.0 | | | |
| (Symrise) | glycol, Avena sativa (oat) | | | | | | | |
| | kernel extract | | | | | | | |
| Dragosan W/O liquid | Polyglyceryl 3-poly- | | | | | | 1.0 | |
| (Symrise) | ricinoleate, sorbitan | | | | | | | |
| | isostearate | | | | | | | |
| Dragosan W/O P | Sorbitan isostearate, | | | | | | 6.0 | |
| (Symrise) | hydrogenated castor oil, | | | | | | | |
| | ceresin, beeswax (Cera | | | | | | | |
| | alba) | | | | | | | |
| Dragosantol (Symrise) | Bisabolol | | | | 0.1 | 0.1 | | |
| Dragoxat EH (Symrise) | Ethylhexyl ethylhexanoate | 3.0 | 3.0 | | 4.0 | | | |
| EDETA B powder | Tetrasodium EDTA | | | | | | | 0.1 |
| (BASF) | | | | | | | | |
| EDETA DB (BASF) | Disodium EDTA | | | | | 0.1 | | |
| Emulsiphos (Symrise) | Potassium cetyl | | 2.0 | | | 1.5 | | |
| | phosphate, hydrogenated | | | | | | | |
| | palm glycerides | | | | | | | |
| Ethanol, 96% | Ethanol | | | | | | | |
| Extrapone green tea | Glycerol, water (aqua), | | 0.2 | | | | | |
| (Symrise) | Camellia sinensis leaf | | | | | | | |
| | extract | | | | | | | |
| Extrapone witch hazel | Propylene glycol, | | | | | | 1.0 | |
| distillate, colorless | Hamamelis virginiana | | | | | | | |
| (Symrise) | (witch hazel), water | | | | | | | |
| | (aqua), Hamamelis | | | | | | | |
| | virginiana (witch hazel) | | | | | | | |
| | extract | | | | | | | |
| Extrapone chamomile | Glycerol, water (aqua), | | 0.5 | | | | | |
| (Symrise) | Chamomilla recutita | | | | | | | |
| | (matricaria) flower extract | | | | | | | |
| Extrapone rosemary | Glycerol, water (aqua), | | 0.3 | | | | | |
| GW (Symrise) | Rosmarinus officinalis | | | | | | | |
| | (rosemary) leaf extract | | | | | | | |
| L-menthyl 3-oxobutyrate | | 0.4 | 0.3 | 0.4 | | 0.5 | 0.4 | 0.5 |
| L-menthyl 3- | | | 0.3 | | 0.5 | | 0.1 | |
| oxopentanoate | | | | | | | | |
| Frescolat ML, | Menthyl lactate | 0.1 | | | | | | 0.5 |
| crystalline, (Symrise) | | | | | | | | |
| Genapol LRO Liquid | Sodium laureth sulphate | | | | | | | 37.0 |
| (Clariant) | | | | | | | | |
| Glycerol, 85% in water | Glycerol | 3.0 | 2.0 | 4.0 | | 4.7 | 2.0 | |
| Urea | Urea | | | 0.5 | | | 1.0 | |
| Hydrolite-5 (1,2- | Pentylene glycol | 1.0 | 0.5 | | 3.0 | 3.0 | | |
| pentanediol) (Symrise) | | | | | | | | |
| Hydroviton (Symrise) | Water, glycerol, sodium | 0.5 | 1.0 | | 1.0 | 1.0 | | 1.0 |
| | lactate, TEA lactate, | | | | | | | |
| | serine, lactic acid, urea, | | | | | | | |
| | sorbitol, sodium chloride, | | | | | | | |
| | lauryl diethylenediamino- | | | | | | | |
| | glycine, lauryl amino- | | | | | | | |
| | propylglycine, allantoin | | | | | | | |
| Isodragol (Symrise) | Triisononanoin | | 2.0 | | | | | |
| Isopropyl palmitate | Isopropyl palmitate | 4.0 | | | | | | |
| (Symrise) | | | | | | | | |
| Karion F (Merck) | Sorbitol | | | | | | 2.0 | |
| Keltrol RD (CP-Kelco) | Xanthan gum | 0.2 | 0.1 | | | | | |
| Keltrol T (Danby- | Xanthan gum | | | | | 0.2 | | |
| Chemie) | | | | | | | | |
| Lanette 16 (Cognis) | Cetyl alcohol | 1.0 | | | | | | |
| Lanette O (Cognis) | Cetearyl alcohol | | 3.0 | | | 1.0 | | |
| Lara Care A-200 (Rahn) | Galactoarabinan | | | 0.3 | | | | |
| Magnesium sulfate | Magnesium sulphate | | | | | | 0.7 | |
| (Merck) | | | | | | | | |
| L-Menthol (Symrise) | Menthol | | | 0.2 | | | | |
| Merquat 550 (Ondeo | Polyquaternium-7 | | | | | | | 0.5 |
| Nalco) | | | | | | | | |
| Sodium benzoate | Sodium benzoate | | | | | | | 0.5 |
| Neo Heliopan 357 | Butyl methoxydibenzoyl- | | | | | 1.0 | | |
| (Symrise) | methane | | | | | | | |
| Neo Heliopan AP | Disodium phenyl dibenz- | | | | | 4.6 | | |
| (Symrise) | imidazole tetrasulphonate | | | | | | | |
| Neo Heliopan AV | Ethylhexyl methoxy- | | | | | 3.0 | | |
| (Symrise) | cinnamate | | | | | | | |
| Neo Heliopan Hydro | Phenylbenzimidazole | | | | | 6.7 | | |
| (Symrise) | sulphonic acid | | | | | | | |
| Neo Heliopan MBC | 4-Methylbenzylidene | | | | | 1.5 | | |
| (Symrise) | camphor | | | | | | | |
| Neo Heliopan OS | Ethylhexyl salicylate | | | | | 5.0 | | |
| (Symrise) | | | | | | | | |
| Neutral oil | Caprylic/capric triglyceride | 6.0 | | | 4.0 | 2.0 | | |
| Oxynex 2004 (Merck) | BHT | | | | | | 0.1 | |
| Paraffin oil 5, grade E | Paraffinum liquidum | | | | 4.0 | | | |
| (Parafluid) | | | | | | | | |
| PCL Liquid 100 | Cetearyl ethylhexanoate | 3.0 | 5.0 | | 7.0 | | | |
| (Symrise) | | | | | | | | |
| PCL Solid (Symrise) | Stearyl heptanoate, | | 2.0 | | | | | |
| | stearyl caprylate | | | | | | | |
| PCL-Liquid (Symrise) | Cetearyl ethylhexanoate, | | | | | | 12.0 | |
| | isopropyl myristate | | | | | | | |
| Pemulen TR-2 | Acrylates/C10-30 alkyl | | | 0.3 | 0.2 | | | |
| (Noveon) | acrylate crosspolymer | | | | | | | |
| 1,2-Propylene glycol | Propylene glycol | | 5.0 | | | | | |
| Sepigel 305 | Polyacrylamide, C13-14 | | | | | | | |
| | isoparaffin, laureth-7 | | | | | | | |
| Sodium chloride | Sodium chloride | | | | | | | 1.0 |
| Sodium hydroxide, 10% | Sodium hydroxide | | 0.3 | 0.6 | 0.4 | | | |
| solution | | | | | | | | |
| Sunflower oil (Wagner) | Helianthus annuus | | | | | | 5.0 | |
| | (sunflower) seed oil | | | | | | | |
| Almond oil (Wagner) | Prunus dulcis | | | | | | 5.0 | |
| Symdiol 68 (Symrise) | 1,2-Hexanediol, caprylyl | 0.5 | | | | | | |
| | glycol | | | | | | | |
| Perfume (Symrise) | Fragrance | 0.3 | 0.3 | 0.3 | 0.2 | 0.4 | 0.4 | 0.5 |
| Tego betaine L7 | Cocamidopropyl betaine | | | | | | | 6.0 |
| (Degussa) | | | | | | | | |
| Tegosoft TN (Degussa) | C12-15 alkyl benzoate | | | 5.0 | | 5.0 | | |
| Triethanolamine | Triethanolamine | | | | | 0.5 | | |
| Retinyl palmitate in oil | Retinyl palmitate | | | | | | 0.2 | |
| (DSM Nutritional | | | | | | | | |
| Products) | | | | | | | | |
| Tocopherol acetate | Tocopheryl acetate | | | 0.5 | | 0.5 | 3.0 | |
| (DSM Nutritional | | | | | | | | |
| Products) | | | | | | | | |
| Water, demineralized | Water (aqua) | to | to | to | to | to | to | to |
| | | make | make | make | make | make | make | make |
| | | up to | up to | up to | up to | up to | up to | up to |
| | | 100 | 100 | 100 | 100 | 100 | 100 | 100 |

### FURTHER EMBODIMENTS

A first embodiment of the present invention is the use of a compound of the formula (I) or (ent-I) or of a blend consisting of two, three or more compounds of the formula (I) or (ent-I)

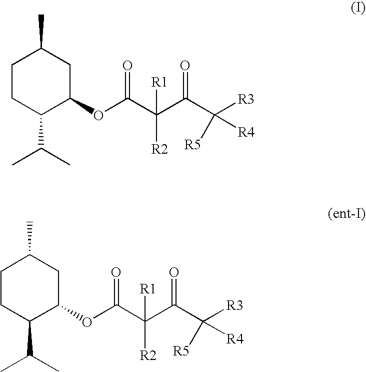

(a) as a cooling substance for non-therapeutic purposes or
(b) for the production of a medicament, wherein in each of the formulae (I) and (ent-I) R1, R2, R3, R4 and R5 mutually independently in each case mean hydrogen or a linear, branched or cyclic, saturated or unsaturated hydrocarbon residue having 1 to 4 carbon atoms.

A second embodiment is a use according to the first embodiment, wherein the compound or at least one of the compounds in the mixture is selected from the group consisting of compounds of the formulae (I) and (ent-I), wherein R1, R2, R3, R4 and R5 mutually independently in each case mean hydrogen or a methyl, ethyl, propyl, cyclopropyl, 2-propyl, 2-propenyl, 1-propenyl, 2-methylpropyl, methyl-2-propenyl, cyclobutyl, 1-butyl, 2-butyl, tert.-butyl or cyclopropylmethyl residue.

A third embodiment is a use according to one of the first two embodiments, wherein the compound or at least one of the compounds in the blend is selected from the group consisting of compounds of the formulae (I) and (ent-I), wherein R1 and R2 mean hydrogen.

A fourth embodiment is a use according to any one of the first three embodiments, wherein the compound or at least one of the compounds in the blend is selected from the group consisting of compounds of the formulae (I) and (ent-I), wherein R3 and R4 mean hydrogen and R5 means hydrogen or methyl.

A fifth embodiment is a use according to any one of the first four embodiments, wherein the compound or at least one of the compounds in the blend is selected from the group consisting of compounds of the formulae (I) and (ent-I), wherein R1, R2, R3 and R4 mean hydrogen and R5 means hydrogen or methyl.

A sixth embodiment is a blend consisting of or comprising:

(a)) a first compound of the formula (I) or (ent-I) as defined in any one of the preceding embodiments and one or more substances selected from the group consisting of:
as a further component of constituent (a), a second compound or two or more further compounds of the formula (I) or (ent-I) as defined in any one of the preceding embodiments,
as constituent (b), a compound or a mixture of two, three or more compounds of the formulae (IIa), (IIb), (IIc), (ent-IIa), (ent-IIb), (ent-IIc)

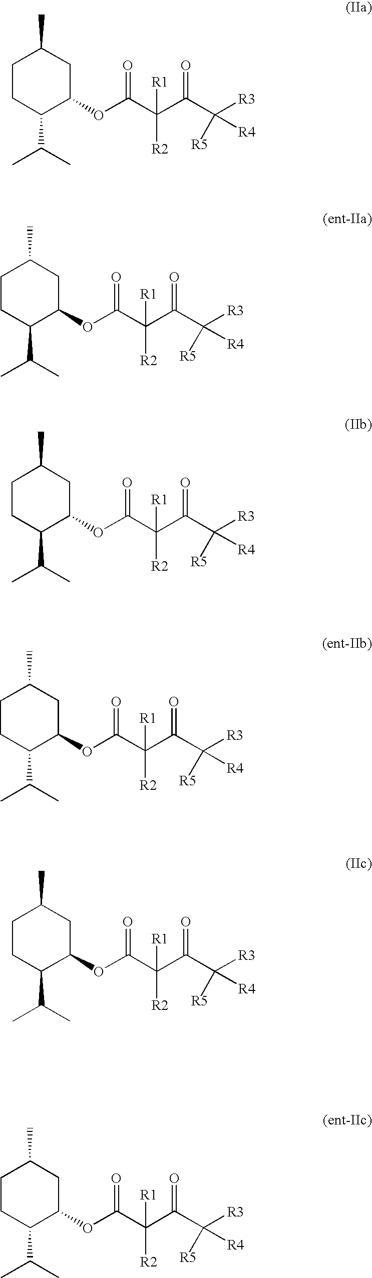

wherein, in each of the formulae (IIa), (IIb), (IIc), (ent-IIa), (ent-IIb) and (ent-IIc), R1, R2, R3, R4 and R5 in each case mutually independently have one of the meanings for the formulae (I) and (ent-I) stated in any one of the preceding embodiments, wherein the particular meanings of R1, R2, R3, R4 and R5 for the compounds of the formulae (I), (ent-I), (IIa), (IIb), (IIc), (ent-IIa), (ent-IIb) and (ent-IIc) present in the blend are in each case mutually independent,
as constituent (c), a one or more further substances having a physiological cooling action, wherein the further substance or one, several or all of the further substances (i) cause(s) a flavor effect or (ii) cause(s) no flavor effect,
as constituent (d), one or more aroma substances without a physiological cooling action
as constituent (e), one or more substances without a physiological cooling action which have a trigeminal or salivatory action,
   wherein, if L-menthyl 3-oxobutyrate is used as the first compound of the formula (I) and a quantity of L-menthyl 3-hydroxybutyrate which causes a physiological cooling action is used as or in constituent (c), the blend does not comprise a reducing agent for L-menthyl 3-oxobutyrate.

A seventh embodiment is a blend according to the sixth embodiment, wherein in one, two, three, more than three or all of the compounds (I), (ent-I), (IIa), (lib), (IIc), (ent-IIa), (ent-IIb) and (ent-IIc) contained in the blend, R1, R2, R3, R4 and R5 have the meanings stated for the formulae (I) and (ent-I) in any one of the preceding embodiments.

An eighth embodiment is a blend according to either the sixth or seventh embodiment, comprising as or in constituent (b) a compound or a mixture of compounds of the formulae (IIa), (IIb) and/or (IIc).

A ninth embodiment is a blend according to any one of the sixth through eighth embodiments, wherein the weight ratio of (a) the entirety of compounds of the formula (I) and (ent-I) to (b) the entirety of compounds of the formulae (IIa), (IIb), (IIc), (ent-IIa), (ent-IIb) and (ent-IIc) is in the range from 200:1 to 4:1, preferably in the range from 100:1 to 10:1, particularly preferably in the range from 100:1 to 20:1.

A tenth embodiment is a blend according to any one of the sixth through ninth embodiments, comprising one or more compounds of the formula (I), wherein the proportion of compounds of the formula (I) to the total weight of compounds of the formulae (I), (ent-I), (IIa), (IIb), (IIc), (ent-IIa), (ent-IIb) and (ent-IIc) amounts to at least 90 wt. %, preferably at least 95 wt. %.

An eleventh embodiment is a blend according to any one of the sixth through tenth embodiments, comprising in or as constituent (c)
one or more substances having a physiological cooling action, wherein these cause no flavor effect and no aroma action or
one, two or more compounds selected from the group consisting of: menthol and menthol derivatives, menthyl ethers, menthyl esters, menthyl carbonates, semiesters of menthol having a dicarboxylic acid and the derivatives thereof, menthane carboxamides, menthone and menthone derivatives, 2,3-dimethyl-2-(2-propyl)-butanoic acid derivatives, isopulegol and the esters thereof, cubebol, synthetic or natural blends containing cubebol, pyrrolidone derivatives of cycloalkyldione derivatives and tetrahydropyrimidin-2-ones.

A twelfth embodiment is a blend according to any one of the sixth through eleventh embodiments, comprising 0.05 to 90 wt. % of constituent (a) and 0.01 to 90 wt. % of constituent (c), relative to the total weight of constituents (a), (b), (c), (d) and (e).

A thirteenth embodiment is a blend according to any one of the sixth through twelfth embodiments, comprising as or in component (d) one or more aroma substances without a physiological cooling action, which causes or cause a flavor impression, a flavor-modulating effect, a trigeminal effect and/or a salivatory stimulus.

A fourteenth embodiment is a blend according to the thirteenth embodiment, comprising as or in component (d) one or more aroma substances without a physiological cooling action, which cause:
one or more flavor impressions from the group consisting of sweet, umami, bitter, salty and sour and/or
one or more flavor-modulating effects from the group consisting of: bitter-masking, umami-enhancing, sweet-enhancing, salt-enhancing and sour-masking and/or
one or more trigeminal stimuli from the group consisting of: spiciness, heat, tingling and pungency and optionally
a salivatory stimulus.

A fifteenth embodiment is a preparation consumed for nutrition or for pleasure or used for oral hygiene or a pharmaceutical or cosmetic preparation comprising a quantity of a compound of the formula (I) or (ent-I) or a blend consisting of two, three or more compounds of the formula (I) or (ent-I) as defined in any one of the first through fifth embodiments which is sufficient to achieve a physiological cooling action on the skin and/or mucous membranes.

A sixteenth embodiment is a preparation according to the fifteenth embodiment comprising or consisting of a blend as claimed in any one of the sixth through fourteenth embodiments, wherein, if in the blend L-menthyl 3-oxobutyrate is used as the first compound of the formula (I) and a quantity of L-menthyl 3-hydroxybutyrate which causes a physiological cooling action is used as or in constituent (c), the preparation does not contain a reducing agent for L-menthyl 3-oxobutyl rate.

A seventeenth embodiment is a preparation according to the fourteenth or fifteenth embodiments, comprising
a total of 0.0001 wt. % to 20 wt. %, preferably 0.0001 to 10 wt. %, particularly preferably 0.001 wt. % to 0.5 wt. % of compounds of the formula (I), relative to the total weight of the preparation,
a total of 0.0000001 to 99.99 wt. %, preferably 10 to 80 wt. % of compounds of the formula (ent-I), constituents (b), (c)) (d) and/or (e) as defined in any one of claims 6 to 14 and further basic materials, auxiliary substances and additives with the exception of water, relative to the total weight of the preparation,
0 to 99.99 wt. % of water, relative to the total weight of the preparation, preferably 5 to 80 wt. %.

An eighteenth embodiment is a preparation consumed for nutrition or pleasure as described in any one of the fifteenth through seventeenth embodiments, selected from the group consisting of: bakery products, confectionery, alcoholic or non-alcoholic beverages, instant beverages, meat products, eggs or egg products, cereal products, dairy products, fruit preparations, vegetable preparations, snacks, fat and oil based products or emulsions thereof, other ready meals and soups, spices, seasoning mixtures, powdered seasonings, semifinished products, nutritional supplements.

A nineteenth embodiment is a preparation used for oral hygiene as described in any one of the fifteenth embodiment through the seventeenth embodiment based on a dental care preparation and selected from the group consisting of: toothpaste, tooth cream, tooth gel, tooth powder, dental cleaning liquid, dental cleaning foam, mouthwash, tooth cream and mouthwash as a 2-in-1 product, hard candies, mouth spray, dental floss and dental care chewing gum.

A twentieth embodiment is a pharmaceutical preparation as described in any one of the fifteenth through seventeenth embodiments, wherein the preparation is an oral pharmaceutical preparation, preferably in the form of capsules, tablets, sugar-coated tablets, granules, pellets, mixtures of solids, dispersions in liquid phases, as emulsions, as powders, as solutions, as pastes or as another swallowable or chewable preparation.

A twenty-first embodiment is a cosmetic preparation as described in any one of the fifteenth through seventeenth embodiments, selected from the group consisting of: soap, synthetic detergent, a liquid washing, shower or bath preparation, emulsion, ointment, paste, gel, oil, toner, balsam, serum, powder, eau de toilette, toilet water, eau de cologne, perfume, wax, stick, roll-on, (pump) spray, aerosol (foaming, non-foaming or post-foaming), foot care product, beard shampoo or care preparation, insect-repellent product, sunscreen product, aftersun preparation, shaving preparation, aftershave preparation, depilatory product, hair care product, conditioner, hair tonic, hair water, hair rinse, hairdressing cream, pomade, permanent wave and setting lotion, hair smoothing product, hair strengthener, styling aid, blonding product, hair lightener, hair conditioner, hair mousse, hair toning product, nail care product, deodorant, antiperspirant, mouthwash, water pick, makeup, makeup remover, eye care preparation, lip cosmetics, lip care preparation, decorative cosmetics, bath articles and mask.

A twenty-second embodiment is a therapeutic or non-therapeutic method for achieving a physiological cooling action on the skin and/or mucous membranes comprising the following step:
application of a quantity sufficient to achieve a physiological cooling action
   (i) of a compound of the formula (I) or (ent-I) or a blend consisting of two, three or more compounds of the formula (I) or (ent-I) as defined in any one of the first through fifth embodiments, or
   (ii) of a blend as described in any one of the sixth through fourteenth embodiments, or
   (iii) of a preparation as described in any one of the fifteenth through twenty-first embodiments
   onto the skin and/or mucous membranes.

1. A blend comprising:
a compound (a) of formula (I) or (ent-I), 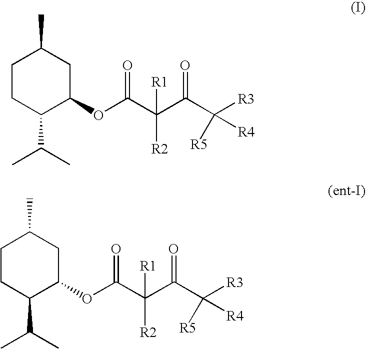
wherein R1, R2, R3, R4, and R5 independently in each case are hydrogen or a linear, branched, or cyclic, saturated or unsaturated hydrocarbon residue having 1 to 4 carbon atoms, and one or more substances selected from the group consisting of:
one or more further compounds of formula (I) or formula (ent-I);
a compound (b) selected from the group consisting of formulae (IIa), (IIb), (IIc), (ent-IIa), (ent-IIb), (ent-IIc), and combinations thereof 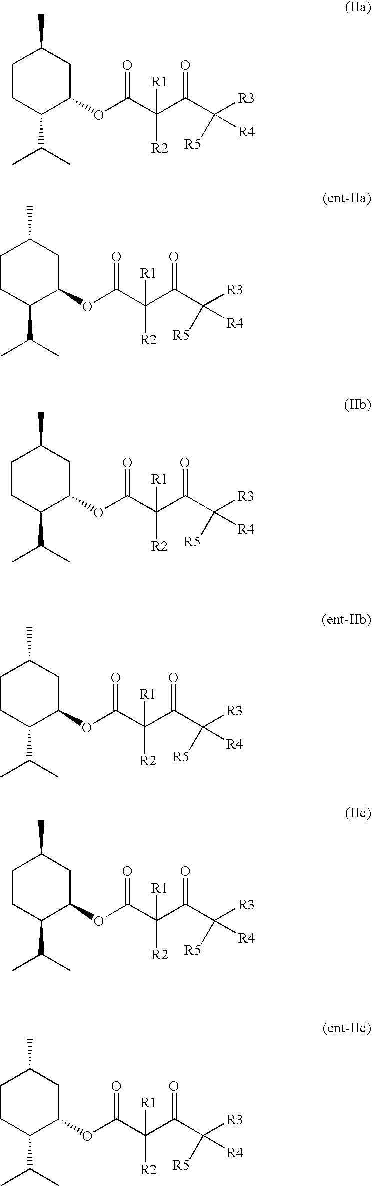
wherein, in each of formulae (IIa), (IIb), (IIc), (ent-IIa), (ent-IIb) and (ent-IIc), R1, R2, R3, R4, and R5 independently in each case are hydrogen or a linear, branched, or cyclic, saturated or unsaturated hydrocarbon residue having 1 to 4 carbon atoms;
one or more further substances (c) having a physiological cooling action, wherein at least one of said one or more further substances optionally causes a flavor effect;
one or more aroma substances (d) without a physiological cooling action; and
one or more substances (e) without a physiological cooling action which have a trigeminal or salivatory action;
with the proviso that if said compound (a) is L-menthyl-3-oxobutyrate and said one or more aroma substances (d) comprises L-menthyl-3-hydroxybutyrate, said blend does not comprise a reducing agent for L-menthyl-3-oxobutyrate.

2. The blend of claim 1, wherein R1, R2, R3, R4, and R5 independently are selected from the group consisting of hydrogen, methyl, ethyl, propyl, cyclopropyl, 2-propyl, 2-propenyl, 1-propenyl, 2-methylpropyl, methyl-2-propenyl, cyclobutyl, 1-butyl, 2-butyl, tert-butyl, and cyclopropylmethyl.

3. The blend of claim 1, wherein said compound (b) comprises a compound of formulae (IIa), (IIb), (IIc), or mixtures thereof.

4. The blend of claim 1, wherein the weight ratio of the total weight of compounds of formula (I) and (ent-I) to the total weight of compounds (b) is in the range of from 200:1 to 4:1.

5. The blend of claim 1, comprising one or more compounds of formula (I), wherein the proportion of compounds of formula (I) to the total weight of compounds of formulae (I), (ent-I), (IIa), (IIb), (IIc), (ent-IIa), (ent-IIb), and (ent-IIc) amounts to at least 90 wt. %.

6. The blend of claim 1, further comprising
one or more substances having a physiological cooling action, wherein said one or more substances causes no flavor effect and no aroma action; or
one or more compounds selected from the group consisting of: menthol and menthol derivatives, menthyl ethers, menthyl esters, menthyl carbonates, semiesters of menthol having a dicarboxylic acid and the derivatives thereof, menthane carboxamides, menthone and menthone derivatives, 2,3-dim ethyl-2-(2-propyl)-butanoic acid derivatives, isopulegol and the esters thereof, cubebol, synthetic or natural blends containing cubebol, pyrrolidone derivatives of cycloalkyldione derivatives and tetrahydropyrimidin-2-ones.

7. The blend of claim 1, comprising 0.05 to 90 wt. % of compound (a) and 0.01 to 90 wt. % of one or more substances (c), relative to the total weight of (a), (b), (c), (d), and (e).

8. The blend of claim 1, wherein said one or more aroma substances (d) causes a flavor impression, a flavor-modulating effect, a trigeminal effect, and/or a salivatory stimulus.

9. The blend of claim 8, wherein one or more aroma substances (d) causes
one or more flavor impressions selected from the group consisting of sweet, umami, bitter, salty and sour; and/or
one or more flavor-modulating effects selected from the group consisting of: bitter-masking, umami-enhancing, sweet-enhancing, salt-enhancing, and sour-masking; and/or
one or more trigeminal stimuli selected from the group consisting of spiciness, heat, tingling, and pungency; and
optionally a salivatory stimulus.

10. A preparation comprising the blend of claim 1, wherein said preparation is consumed for nutrition or for pleasure or used for oral hygiene or a pharmaceutical or cosmetic preparation, and which is sufficient to achieve a physiological cooling action on the skin and/or mucous membranes.

11. The preparation of claim 10, wherein said preparation comprises
a total of 0.0001 wt. % to 20 wt. % of compounds of formula (I), relative to the total weight of the preparation;
a total of 0.0000001 to 99.99 wt. % of compounds of formula (ent-I), compound (b), one or more substances (c), one or more aroma substances (d), one or more substances (e), and further basic materials, auxiliary substances, and additives with the exception of water, relative to the total weight of the preparation; and
0 to 99.99 wt. % of water, relative to the total weight of the preparation.

12. A therapeutic or non-therapeutic method for achieving a physiological cooling action on skin and/or mucous membranes, comprising applying a quantity sufficient to achieve a physiological cooling action of a compound selected from the group consisting of:
(i) a compound selected from the group consisting of compounds of formula (I), formula (ent-I), and combinations thereof 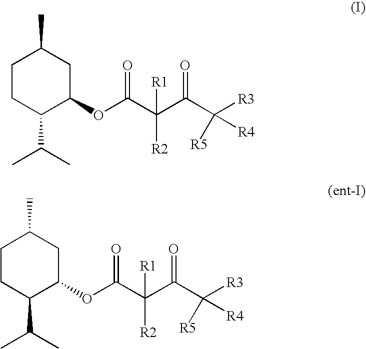
wherein
R1, R2, R3, R4, and R5 independently in each case are hydrogen or a linear, branched, or cyclic, saturated or unsaturated hydrocarbon residue having 1 to 4 carbon atoms; or
(ii) a blend comprising:
compound (a) of the formula (I) or (ent-I), 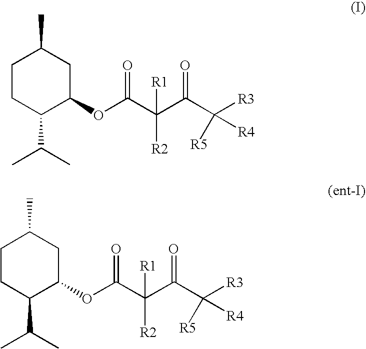
wherein R1, R2, R3, R4, and R5 independently in each case are hydrogen or a linear, branched, or cyclic, saturated or unsaturated hydrocarbon residue having 1 to 4 carbon atoms, and one or more substances selected from the group consisting of:
one or more further compounds of formula (I) or formula (ent-I);
a compound (b) selected from the group consisting of formulae (IIa), (IIb), (IIc), (ent-IIa), (ent-IIb), (ent-IIc), and combinations thereof 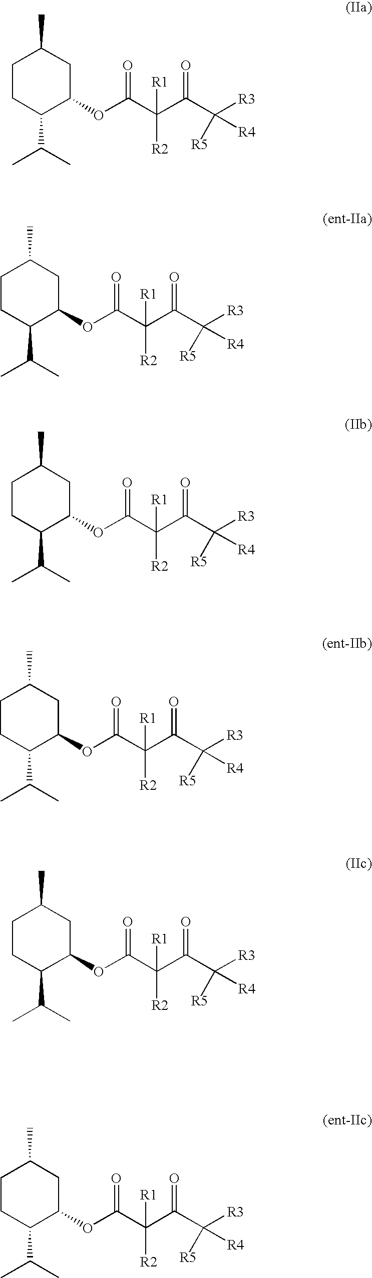
wherein, in each of formulae (IIa), (IIb), (IIc), (ent-IIa), (ent-IIb) and (ent-IIc), R1, R2, R3, R4, and R5 independently in each case are hydrogen or a linear, branched, or cyclic, saturated or unsaturated hydrocarbon residue having 1 to 4 carbon atoms;
one or more further substances (c) having a physiological cooling action, is wherein at least one of said one or more further substances optionally causes a flavor effect;
one or more aroma substances (d) without a physiological cooling action; and
one or more substances (e) without a physiological cooling action which have a trigeminal or salivatory action;
with the proviso that if said compound (a) is L-menthyl-3-oxobutyrate and said one or more aroma substances (d) comprises L-menthyl-3-hydroxybutyrate, said blend does not comprise a reducing agent for L-menthyl-3-oxobutyrate; or
(iii) a preparation comprising a compound selected from the group consisting of compounds of formula (I), formula (ent-I), and combinations thereof 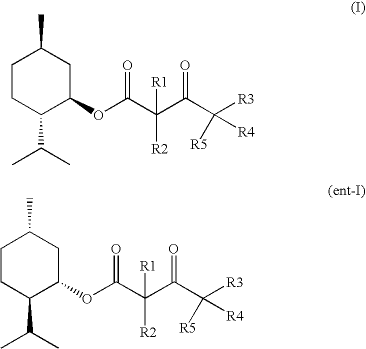
wherein
R1, R2, R3, R4, and R5 independently in each case are hydrogen or a linear, branched, or cyclic, saturated or unsaturated hydrocarbon residue having 1 to 4 carbon atoms and wherein said preparation is consumed for nutrition or for pleasure or used for oral hygiene or a pharmaceutical or cosmetic preparation and which is sufficient to achieve a physiological cooling action on the skin and/or mucous membranes;
onto the skin and/or mucous membranes.

13. The method of claim 12, wherein for (i), R1, R2, R3, R4, and R5 independently are selected from the group consisting of hydrogen, methyl, ethyl, propyl, cyclopropyl, 2-propyl, 2-propenyl, 1-propenyl, 2-methylpropyl, methyl-2-propenyl, cyclobutyl, 1-butyl, 2-butyl, tert-butyl, and cyclopropylmethyl.

14. The method of claim 12, wherein for (i), R1 and R2 are hydrogen.

15. The method of claim 12, wherein for (i), R3 and R4 are hydrogen and R5 is hydrogen or methyl.

16. The method of claim 12, wherein for (i), R1, R2, R3 and R4 are hydrogen and R5 is hydrogen or methyl.

17. The method of claim 12, wherein (iii) is selected from the group consisting of bakery products, confectionery, alcoholic beverages, non-alcoholic beverages, instant beverages, meat products, eggs, egg products, cereal products, dairy products, fruit preparations, vegetable preparations, snacks, fat and oil based products and emulsions thereof, ready meals and soups, spices, seasoning mixtures, powdered seasonings, semifinished products, and nutritional supplements.

18. The method of claim 12, wherein (iii) is a dental care preparation selected from the group consisting of toothpaste, tooth cream, tooth gel, tooth powder, dental cleaning liquid, dental cleaning foam, mouthwash, tooth cream and mouthwash as a 2-in-1 product, hard candies, mouth spray, dental floss, and dental care chewing gum.

19. The method of claim 12, wherein (iii) is an oral pharmaceutical preparation.

20. The method of claim 12, wherein (iii) is selected from the group consisting of soap, synthetic detergent, a liquid washing, shower or bath preparation, emulsion, ointment, paste, gel, oil, toner, balsam, serum, powder, eau de toilette, toilet water, eau de cologne, perfume, wax, stick, roll-on, (pump) spray, aerosol (foaming, non-foaming or post-foaming), foot care product, beard shampoo, beard care preparation, insect-repellent product, sunscreen product, aftersun preparation, shaving preparation, aftershave preparation, depilatory product, hair care product, conditioner, hair tonic, hair water, hair rinse, hairdressing cream, pomade, permanent wave and setting lotion, hair smoothing product, hair strengthener, styling aid, blonding product, hair lightener, hair conditioner, hair mousse, hair toning product, nail care product, deodorant, antiperspirant, mouthwash, water pick, makeup, makeup remover, eye care preparation, lip cosmetics, lip care preparation, decorative cosmetics, bath articles, and mask.
